(19) United States
(12) Patent Application Publication
(10) Pub. No.: US 2021/0347793 A1
(43) Pub. Date: Nov. 11, 2021

(54) CALCIUM OXALATE CRYSTALLIZATION INHIBITORS FOR RENAL DISORDERS

(71) Applicant: INOSITEC AG, Zürich (CH)

(72) Inventors: Mattias Emanuel IVARSSON, Zürich (CH); Jean-Christophe LEROUX, Zürich (CH); Anna KLETZMAYR, Zürich (CH)

(73) Assignee: INOSITEC AG, Zürich (CH)

(21) Appl. No.: 17/276,837

(22) PCT Filed: Sep. 18, 2019
(86) PCT No.: PCT/EP2019/074986

§ 371 Date: Mar. 17, 2021

(30) Foreign Application Priority Data
Sep. 18, 2018 (EP) 18195316.7
Mar. 20, 2019 (EP) 19164195.0

(51) Int. Cl. C07F 9/09

(52) U.S. Cl. CPC C07F 9/09

(57) ABSTRACT

The present invention relates to inositol derivatives comprising two or more cyclohexanolpentakisester moieties linked by a common central linker and their use in therapy or prevention of a condition related to pathological crystallization. The invention further relates to useful intermediates in the synthesis of the compound of the invention.

### BACKGROUND OF THE INVENTION

Kidney stones are mineral deposits in the renal tissue, which form due to supersaturation of the urine with a certain mineral. The prevalence of kidney stones is high with rates up to 14.8%, and a drastic risk of recurrence. Moreover, incidence is on the rise due to changes in lifestyle, diet and the enhanced occurrence of associated risk factors, such as obesity, diabetes and hypertension.

Underlying causes that lead to the formation of kidney stones are diverse. However, one common constituent of the majority of kidney stones is calcium oxalate (CaOx). CaOx crystallization and stone formation in the kidney can physically obstruct the tubules, as well as elicit inflammation and fibrosis of the tissue, together with impairing the organ's function, creating the risk of developing chronic kidney disease and end-stage renal disease.

Another calcium precipitate of clinical importance in the kidney is calcium phosphate. Kidney stones composed predominantly (50% or more) of calcium phosphate constitute up to 10% of all stones and 15%-20% of calcium stones, 80% of which are composed of calcium oxalate. Calcium phosphate is a minor component of up to 30% of calcium oxalate stones as well.

Different diseases, such as hyperoxaluria, Dent disease or Bartter's syndrome, can lead to CaOx kidney stone formation. In the case of primary hyperoxaluria for example, oxalate is produced in excess due to absence or malfunction of liver proteins involved in oxalate metabolism. The high oxalate load in urine causes CaOx crystallization in the renal tubes, kidney stone formation and hence damage of the tissue. Patients suffer from kidney failure and end-stage renal disease at a young age. Due to limited treatment options 80% of patients need combined liver-kidney transplantation at the age of 30.

To date therapeutic options for kidney stone patients are merely palliative and far from disease modifying, thus leaving the patient at risk for stone recurrence and severe complications, such as chronic kidney disease and end stage renal disease. Existing stones can be removed surgically, accompanied by medical management of the pain associated with kidney stones. However, recurrence rate is high and preventive measurements are limited. Suggested measures include high fluid intake, adapted nutrition and thiazides for patients with high urinary calcium levels.

Although CaOx crystallization inhibitors (such as citrate or phosphate, among others) have been proposed to be used to prevent kidney stone formation, limited efficacy and unwanted side effects limit their application in the clinic. Citrate has shown some efficacy as a preventive measurement in recurrent CaOx stone patients, however its mode of action remains unclear.

Taken together, a pharmaceutical agent capable of decreasing the propensity for CaOx crystal nucleation, growth and adherence to renal epithelial cells, and ultimately reducing damage of the kidneys independent of the underlying cause, would be significant therapeutic value. A potent inhibitor of CaOx crystallization in urine would be applicable in a variety of disorders associated with kidney stone formation and could decrease the risk of further, severe complications.

Based on the above-mentioned state of the art, the objective of the present invention is to provide pharmaceutical compounds for treating or preventing calcium oxalate and/or calcium phosphate crystallization in conditions related to pathological calcium oxalate crystallization. This objective is attained by the subject-matter of the independent claims of the present specification.

### DESCRIPTION

### Summary of the Invention

A first aspect of the invention relates to a compound comprising two or more cyclohexanolpentakisester moieties described by a general formula (I) linked by a common central linker L,

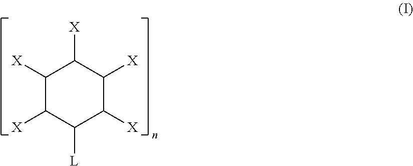

wherein X is selected from OPO₃²⁻, OPSO₂²⁻, or OSO₃⁻, L is a common central linker to which n individual moieties characterized by the formula in brackets are attached and n is an integer from 2 to 10.

In certain embodiments, the compound is described by the general formula (II), (III) or (IV)

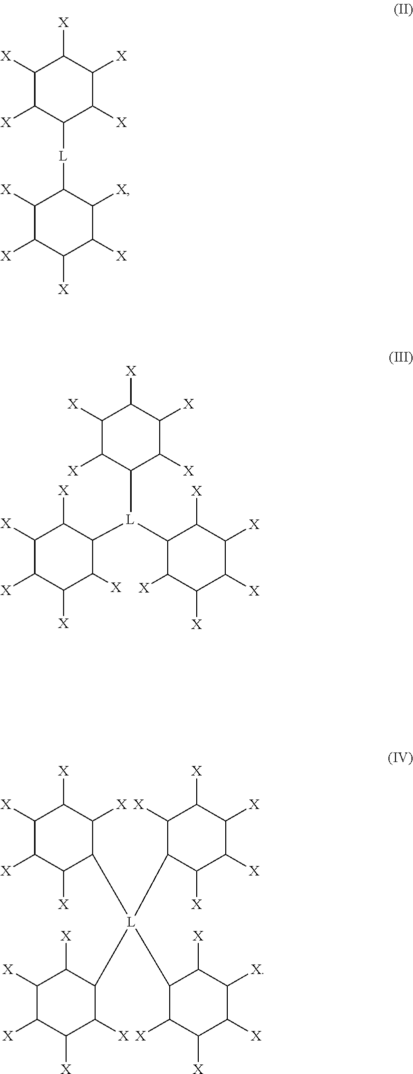

It is understood that any compounds of the invention, where defined herein as a negatively charged compound, will be associated with pharmaceutically acceptable counterions.

A second aspect of the invention relates to the use of a compound as specified herein for treatment of a pathology associated with crystallization, or formation, of calcium and/or magnesium precipitates.

Likewise, the invention relates to the use of a compound as specified herein for treatment of a pathology associated with crystallization, or formation, of oxalate precipitates.

Another aspect of the invention relates to a pharmaceutical composition comprising a therapeutically effective amount of at least one of the compounds of the present invention or a pharmaceutically acceptable salt thereof and at least one pharmaceutically acceptable carrier, diluent or excipient.

### Terms and Definitions

The term C₁-C₄ alkyl in the context of the present specification signifies a saturated linear or branched hydrocarbon having 1, 2, 3 or 4 carbon atoms, wherein in certain embodiments one carbon-carbon bond may be unsaturated and one CH₂ moiety may be exchanged for oxygen (ether bridge) or nitrogen (NH, or NR with R being methyl, ethyl, or n- or iso-propyl; amino bridge). Non-limiting examples for a C₁-C₄ alkyl are methyl, ethyl, propyl, prop-2-enyl, n-butyl, 2-methylpropyl, tert-butyl, but-3-enyl, prop-2-inyl and but-3-inyl. In certain embodiments, a C₁-C₄ alkyl is a methyl, ethyl, propyl or butyl moiety.

A C₁-C₆ alkyl in the context of the present specification signifies a saturated linear or branched hydrocarbon having 1, 2, 3, 4, 5 or 6 carbon atoms, wherein one carbon-carbon bond may be unsaturated and one CH₂ moiety may be exchanged for oxygen (ether bridge) or nitrogen (NH, or NR with R being methyl, ethyl, or n- or iso-propyl; amino bridge). Non-limiting examples for a C₁-C₆ alkyl include the examples given for C₁-C₄ alkyl above, and additionally 3-methylbut-2-enyl, 2-methylbut-3-enyl, 3-methylbut-3-enyl, n-pentyl, 2-methylbutyl, 3-methylbutyl, 1,1-dimethylpropyl, 1,2-dimethylpropyl, 1,2-dimethylpropyl, pent-4-inyl, 3-methyl-2-pentyl, and 4-methyl-2-pentyl. In certain embodiments, a C₅ alkyl is a pentyl or cyclopentyl moiety and a C₆ alkyl is a hexyl or cyclohexyl moiety.

The term unsubstituted Cₙ alkyl when used herein in the narrowest sense relates to the moiety —CₙH₂ₙ— if used as a bridge between moieties of the molecule, or —CₙH₂ₙ₊₁ if used in the context of a terminal moiety. It may still contain fewer H atoms if a cyclical structure or one or more (non-aromatic) double bonds are present.

The terms unsubstituted Cₙ alkyl and substituted Cₙ alkyl include a linear alkyl comprising or being linked to a cyclical structure, for example a cyclopropane, cyclobutane, cyclopentane or cyclohexane moiety, unsubstituted or substituted depending on the annotation or the context of mention, having linear alkyl substitutions. The total number of carbon and—where appropriate—N, O or other hetero atom in the linear chain or cyclical structure adds up to n.

Where used in the context of chemical formulae, the following abbreviations may be used: Me is methyl CH₃, Et is ethyl —CH₂CH₃, Prop is propyl —(CH₂)₂CH₃ (n-propyl, n-pr) or —CH(CH₃)₂ (iso-propyl, i-pr), but is butyl —C₄H₉, —(CH₂)₃CH₃, —CHCH₃CH₂CH₃, —CH₂CH(CH₃)₂ or —C(CH₃)₃.

The term C₄-C₇ cycloalkyl in the context of the present specification relates to a saturated hydrocarbon ring having 4, 5, 6 or 7 carbon atoms, wherein in certain embodiments, one carbon-carbon bond may be unsaturated and/or one CH₂ moiety may be exchanged for oxygen (ether bridge) or nitrogen (NH, or NR with R being methyl, ethyl, or propyl; amino bridge). Non-limiting examples for a C₄-C₇ cycloalkyl moieties include cyclopropanyl (—C₄H₇), cyclopentenyl (C₅H₉), and cyclohexenyl (C₆H₁₁) moieties. In certain embodiments, a cycloalkyl is substituted by one C₁ to C₄ unsubstituted alkyl moiety. In certain embodiments, a cycloalkyl is substituted by more than one C₁ to C₄ unsubstituted alkyl moieties.

The term substituted alkyl in its broadest sense refers to an alkyl as defined above in the broadest sense, which is covalently linked to an atom that is not carbon or hydrogen, particularly to an atom selected from N, O, F, B, Si, P, S, Cl, Br and I, which itself may be—if applicable—linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitrile CN, isonitrile NC, cyanate CNO, isocyanate NCO, thiocyanate CNS, isothiocyanate NCS, fluoride F, chloride Cl, bromide Br, iodide I, phosphonate PO₃H₂, PO₃R₂, phosphate OPO₃H₂ and OPO₃R₂, sulfhydryl SH, sulphalkyl SR, sulfoxide SOR, sulfonyl SO₂R, sulphanylamide SO₂NHR, sulphate SO₃H and sulphate ester SO₃R, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified.

The term amino substituted alkyl or hydroxyl substituted alkyl refers to an alkyl according to the above definition that is modified by one or several amine or hydroxyl groups NH₂, NHR, NR₂ or OH, wherein the R substituent as used in the current paragraph, different from other uses assigned to R in the body of the specification, is itself an unsubstituted or substituted C₁ to C₁₂ alkyl in its broadest sense, and in a narrower sense, R is methyl, ethyl or propyl unless otherwise specified. An alkyl having more than one carbon may comprise more than one amine or hydroxyl. Unless otherwise specified, the term “substituted alkyl” refers to alkyl in which each C is only substituted by at most one amine or hydroxyl group, in addition to bonds to the alkyl chain, terminal methyl, or hydrogen.

The term carboxyl substituted alkyl refers to an alkyl according to the above definition that is modified by one or several carboxyl groups COOH, or derivatives thereof, particularly carboxylamides CONH₂, CONHR and CONR₂, or carboxylic esters COOR, with R having the meaning as laid out in the preceding paragraph and different from other meanings assigned to R in the body of this specification.

Non-limiting examples of amino-substituted alkyl include —CH₂NH₂, —CH₂NHMe, —CH₂NHEt, —CH₂CH₂NH₂, —CH₂CH₂NHMe, —CH₂CH₂NHEt, —(CH₂)₃NH₂, —(CH₂)₃NHMe, —(CH₂)₃NHEt, —CH₂CH(NH₂)CH₃, —CH₂CH(NHMe)CH₃, —CH₂CH(NHEt)CH₃, —(CH₂)₃CH₂NH₂, —(CH₂)₃CH₂NHMe, —(CH₂)₃CH₂NHEt, —CH(CH₂NH₂)CH₂CH₃, —CH(CH₂NHMe)CH₂CH₃, —CH(CH₂NHEt)CH₂CH₃, —CH₂CH(CH₂NH₂)CH₃, —CH₂CH(CH₂NHMe)CH₃, —CH₂CH(CH₂NHEt)CH₃, —CH(NH₂)(CH₂)₂NH₂, —CH(NHMe)(CH₂)₂NHMe, —CH(NHEt)(CH₂)₂NHEt, —CH₂CH(NH₂)CH₂NH₂, —CH₂CH(NHMe)CH₂NHMe, —CH₂CH(NHEt)CH₂NHEt, —CH₂CH(NH₂)(CH₂)₂NH₂, —CH₂CH(NHMe)(CH₂)₂NHMe, —CH₂CH(NHEt)(CH₂)₂NHEt, —CH₂CH(CH₂NH₂)₂, —CH₂CH(CH₂NHMe)₂ and —CH₂CH(CH₂NHEt)₂ for terminal moieties and —CH₂CHNH₂—, —CH₂CHNHMe-, —CH₂CHNHEt- for an amino substituted alkyl moiety bridging two other moieties.

Non-limiting examples of hydroxy-substituted alkyl include —CH₂OH, —(CH₂)₂OH, —(CH₂)₃OH, —CH₂CH(OH)CH₃, —(CH₂)₄OH, —CH(CH₂OH)CH₂CH₃, —CH₂CH(CH₂OH)CH₃, —CH(OH)(CH₂)₂OH, —CH₂CH(OH)CH₂OH, —CH₂CH(OH)(CH₂)₂OH and —CH₂CH(CH₂OH)₂ for terminal moieties and —CHOH—, —CH₂CHOH—, —CH₂CH(OH)CH₂—, —(CH₂)₂CHOHCH₂—, —CH(CH₂OH)CH₂CH₂—, —CH₂CH(CH₂OH)CH₂—, —CH(OH)(CH₂CHOH—, —CH₂CH(OH)CH₂OH, —CH₂CH(OH)(CH₂)₂OH and —CH₂CHCH₂OHCHOH— for a hydroxyl substituted alkyl moiety bridging two other moieties.

The term halogen-substituted alkyl refers to an alkyl according to the above definition that is modified by one or several halogen atoms selected (independently) from F, Cl, Br, I.

The term fluoro substituted alkyl refers to an alkyl according to the above definition that is modified by one or several fluoride groups F. Non-limiting examples of fluoro-substituted alkyl include —CH₂F, —CHF₂, —CF₃, —(CH₂)₂F, —(CHF)₂H, —(CHF)₂F, —C₂F₅, —(CH₂)₃F, —(CHF)₃H, —(CHF)₃F, —C₃F₇, —(CH₂)₄F, —(CHF)₄H, —(CHF)₄F and —C₄F₉.

Non-limiting examples of hydroxyl- and fluoro-substituted alkyl include —CHFCH₂OH, —CF₂CH₂OH, —(CHF)₂CH₂OH, —(CF₂)₂CH₂OH, —(CHF)₃CH₂OH, —(CF₂)₃CH₂OH, —(CH₂)₃OH, —CF₂CH(OH)CH₃, —CF₂CH(OH)CF₃, —CF(CH₂OH)CHFCH₃, and —CF(CH₂OH)CHFCF₃.

The term aryl in the context of the present specification signifies a cyclic aromatic C₅-C₁₀ hydrocarbon that may comprise a heteroatom (e.g. N, O, S). Examples of aryl include, without being restricted to, phenyl and naphthyl, and any heteroaryl. A heteroaryl is an aryl that comprises one or several nitrogen, oxygen and/or sulphur atoms. Examples for heteroaryl include, without being restricted to, pyrrole, thiophene, furan, imidazole, pyrazole, thiazole, oxazole, pyridine, pyrimidine, thiazin, quinoline, benzofuran and indole. An aryl or a heteroaryl in the context of the specification additionally may be substituted by one or more alkyl groups.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624).

A first aspect of the invention relates to a compound comprising two or more cyclohexanolpentakisester moieties described by a general formula (I) linked by a common central linker L,

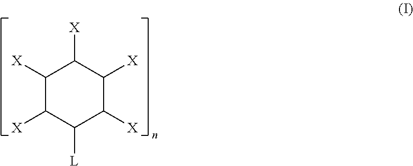

wherein X is selected from OPO₃²⁻, OPSO₂²⁻, or OSO₃⁻, L is a common central linker to which n individual moieties characterized by the formula in brackets are attached and n is an integer from 2 to 10.

The straight lines in formula I and elsewhere herein are meant to indicate that the stereochemistry of the individual ring carbon atoms is undefined. The formula is meant to encompass any diastereomer.

In certain embodiments n has a value from 2 to 8. In certain embodiments, n has a value of 2. In certain embodiments n has a value of 3. In certain embodiments n has a value of 4. In certain embodiments n has a value of 6. In certain embodiments n has a value of 8.

In certain particular embodiments, n is selected from 2, 3 and 4, In certain embodiments, the central linker moiety L comprises or consists of a linear or branched poly(ethylene glycol) or polyglycerol. In certain embodiments, the central linker moiety L comprises or consists of a linear or branched alkyl.

In certain embodiments, the linking moiety L has a molecular weight <1000 g/mol, particularly <700 g/mol, more particularly <500 g/mol, or even <400 g/mol.

In certain embodiments, the compound of the invention consists of the central linker L as defined above, and the two or more moieties described by the general formula (I).

In certain embodiments, any of the X of any moiety (I) comprised in the compound according to the invention is selected independently from any other X from OPO₃²⁻, OPSO₂²⁻, or OSO₃⁻.

In certain embodiments, all of the X of any particular moiety (I) comprised in the compound according to the invention are the same, and are selected from OPO₃²⁻, OPSO₂²⁻, or OSO₃⁻.

In certain embodiments, all of the X of all moieties (I) comprised in the compound according to the invention are OPO₃²⁻.

In certain embodiments, all of the X of all moieties (I) comprised in the compound according to the invention are OPSO₂²⁻.

In certain embodiments, all of the X of all moieties (I) comprised in the compound according to the invention are OSO₃⁻.

In certain embodiments, the linking moiety L comprises or consists of a linear or branched poly(ethylene glycol) or polyglycerol having a molar mass <1000 g/mol, particularly <700 g/mol, more particularly <500 g/mol, or even <400 g/mol.

In certain embodiments, the linking moiety comprises a central core A, to which polyglycerol or poly(ethylene glycol) chains separating the cyclohexanolester moieties of formula (I) are attached.

In certain embodiments, the central core A, if present, can be selected from a substituted or unsubstituted C₁ to C₄ alkyl, a substituted or unsubstituted C₄ to C₇ cycloalkyl, or a substituted or unsubstituted five- or six-membered aryl.

The linking moiety is generally a simple, metabolically compatible linker designed to facilitate assembly of two or more cyclohexanolpentakisester moieties in the compound at relatively low cost and synthetic ease. In certain embodiments, the linker moiety may be a hydrocarbon substituted by substituents other than hydrogen, for example by one or more substituents selected from oxygen, nitrogen, fluorine or other substituents known in the art of medicinal chemistry to modulate the absorption, distribution, metabolic processing, excretion or toxicity qualities of a compound.

For embodiments wherein the linking moiety L is or comprises a substituted C₁ to C₄ alkyl, a substituted C₄ to C₇ cycloalkyl, or a substituted five- or six-membered aryl, the substitution may be one or several functions comprising oxygen (by non-limiting example: —OH or ═O), one or several functions comprising nitrogen (by non-limiting example: NH₂, NHR^{N}, NRN₂, with RN selected from unsubstituted C₁ to C₄ alkyl), one or several fluorine substituents, or any combination of oxygen, nitrogen or fluorine substituents.

In certain embodiments of the compound described by a general formula (I), the linking moiety L is a peptide linker. The moieties described by formula (I) can be covalently linked to the side chain of a proteinogenic amino acid, particularly one having a side chain facilitating chemical attachment of the cyclohexanolester moieties. Examples of such amino acids having a favourable attachment functionality are, by non-limiting example, serine and threonine.

In certain embodiments, any one of the cyclohexanolpentakisester moieties described by the general formula (I) is independently selected from (Ia) and (Ib)

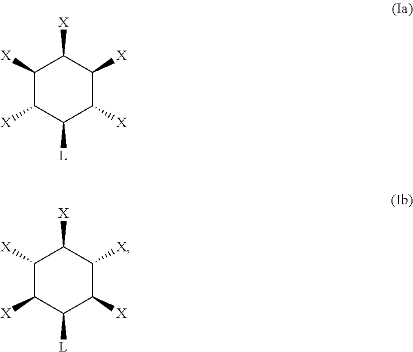

wherein each X (independently) has the meaning outlined above, and wherein L is a linking moiety.

In certain embodiments, the compound is described by the general formula (II),

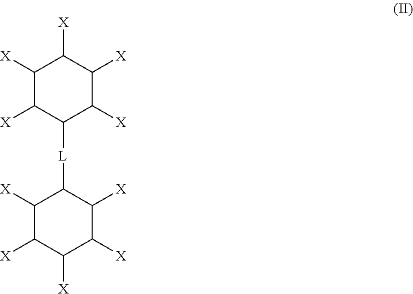

wherein each X independently from any other X can be OPO₃²⁻, OPSO₂²⁻, OSO₃⁻ or CO₂⁻, and wherein L is a common central linker (also referred to as a “linking moiety” herein) as specified above.

In certain embodiments, the compound is described by the general formula (IIi), (IIii), or (IIiii):

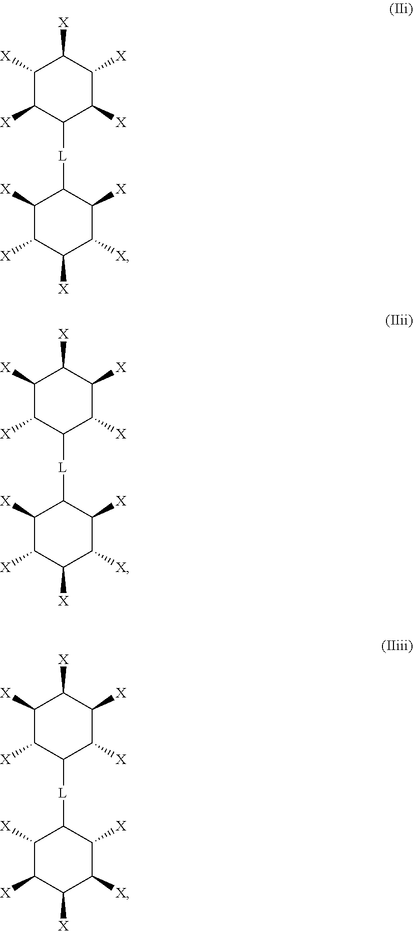

wherein each X independently from any other X can be OPO₃²⁻, OPSO₂²⁻, OSO₃⁻ or CO₂⁻, and wherein L is a common central linker (also referred to as a “linking moiety” herein) as specified above.

In certain embodiments, the compound is described by the general formula (II), (IIi), (IIii), or (IIiii), wherein the linking moiety L is selected from a poly(ethylene glycol) described by a formula —(O—CH₂—CH₂)ₘ—O—, and m has a value from a value between 1 and 12. In particular embodiments, n is selected from 2, 3, or 4.

In certain embodiments the linking moiety L is selected from a polyglycerol described by a formula —(O—CH₂—CH(OH)—CH₂)ₘ—O—, and m has a value from a value between 1 and 12. In particular embodiments, m is selected from 2, 3, or 4.

In certain embodiments, all moieties described by the general formula (I) are either (la) or (Ib).

In certain embodiments, the compound is described by formula (II), (IIi), (IIii), or (IIiii), each X is OPO₃²⁻ and the linking moiety L is a linear poly(ethylene glycol) or polyglycerol.

In certain embodiments, the compound described by formula (II), (IIi), (IIii), or (IIiii) has a molecular weight between 1000 g/mol to 1500 g/mol.

In certain embodiments, the compound is described by formula (II), (IIi), (IIii), or (IIiii), each X is OPO₃²⁻, the linking moiety L is a linear poly(ethylene glycol) or polyglycerol and the compound has a molecular weight between 1000 g/mol to 1500 g/mol.

In certain embodiments, the compound is described by the general formula (IIiv), (IIv), or (IIvi):

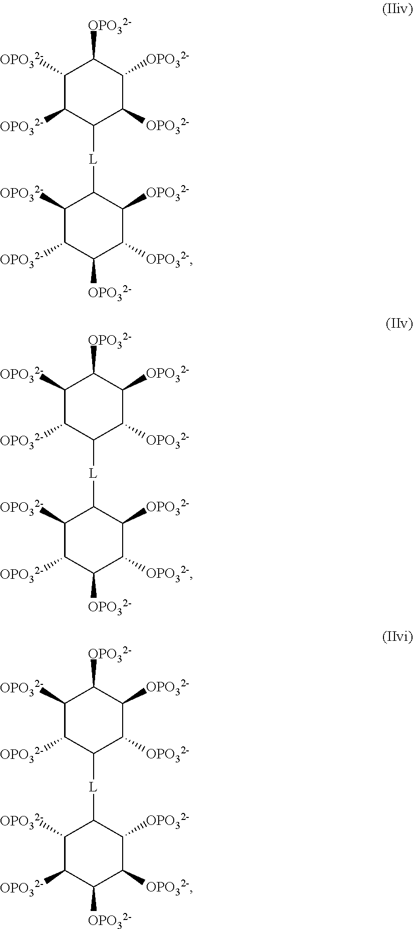

wherein L is a common central linker (also referred to as a “linking moiety” herein) as specified above. In certain embodiments, the compound is described by the general formula (IIvii), (IIviii), or (IIix):

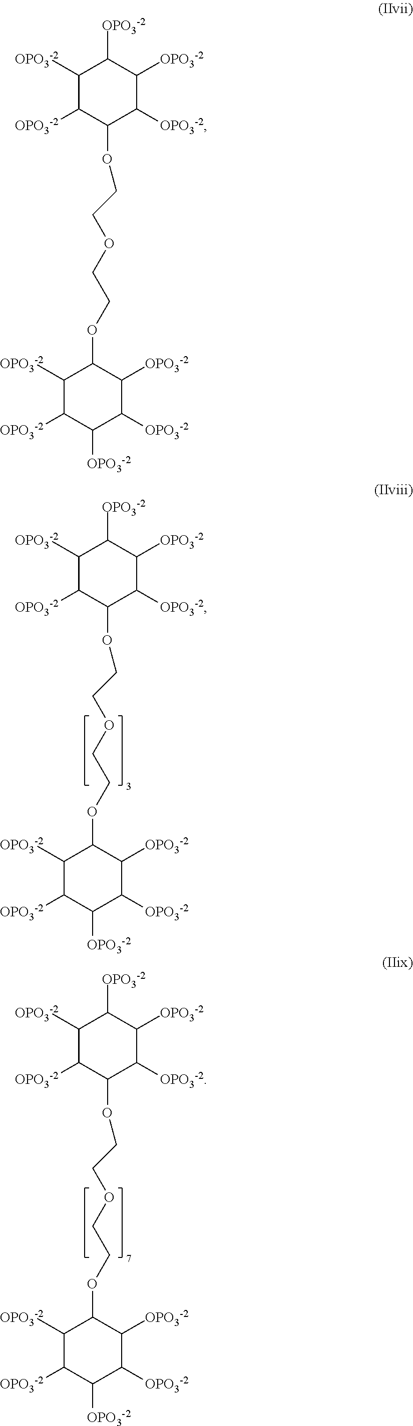

In certain embodiments, the compound is described by formula (IIa), (IIb), (IIc) or (IId):

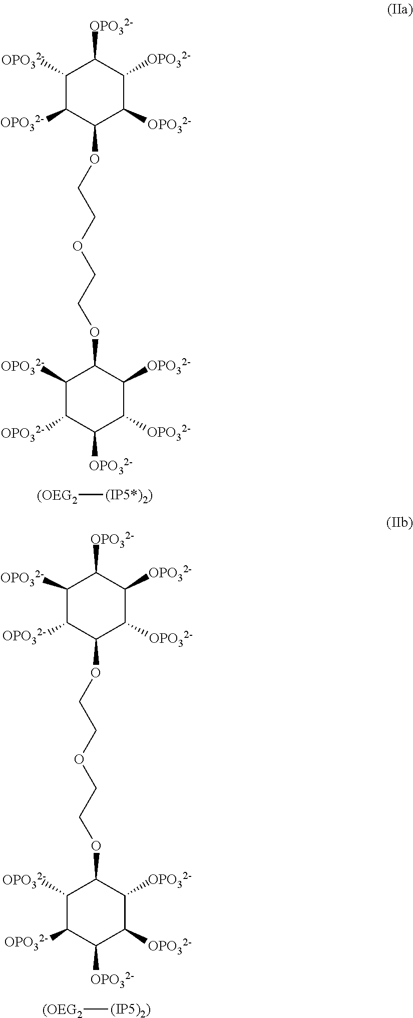

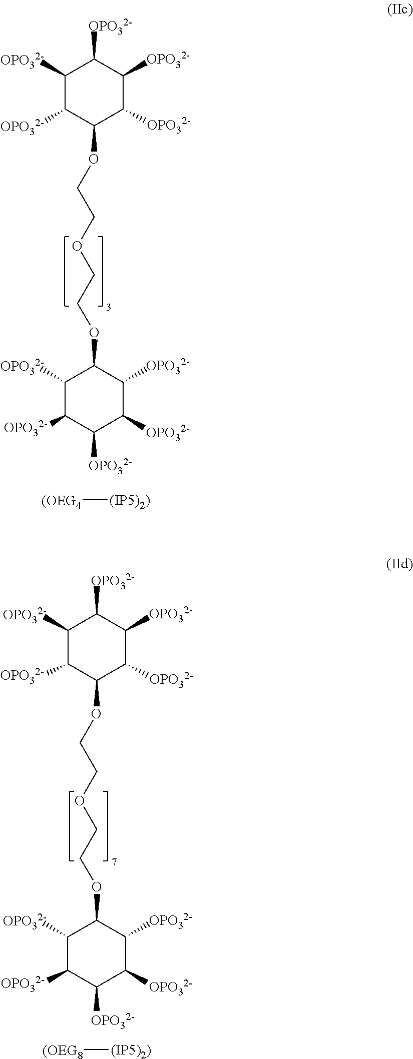

In certain embodiments, the compound is described by formula (IIe) or (IIf):

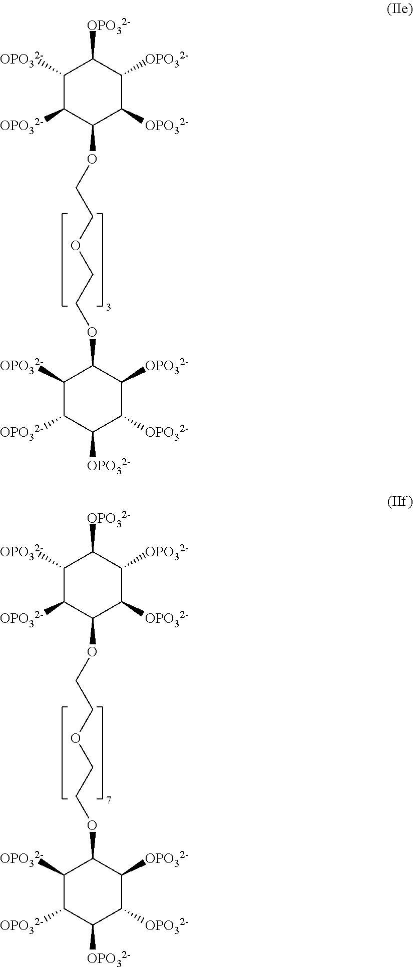

In certain embodiments, the compounds (IIa) (OEG₂-(IP5*)₂) and (IIb) (OEG₂-(IP5)₂) have a molecular mass of 1204.04 g/mol. In one embodiment, the compounds (IIc) (PO₁-1) and (IIe) have a molecular mass of 1298.15 g/mol. In one embodiment, the compounds (IId) (OEG₈-(IP5)₂) and (IIf) have a molecular mass of 1474.36 g/mol.

In certain embodiments, the compound is described by formula (IIk), (IIm), or (IIn):

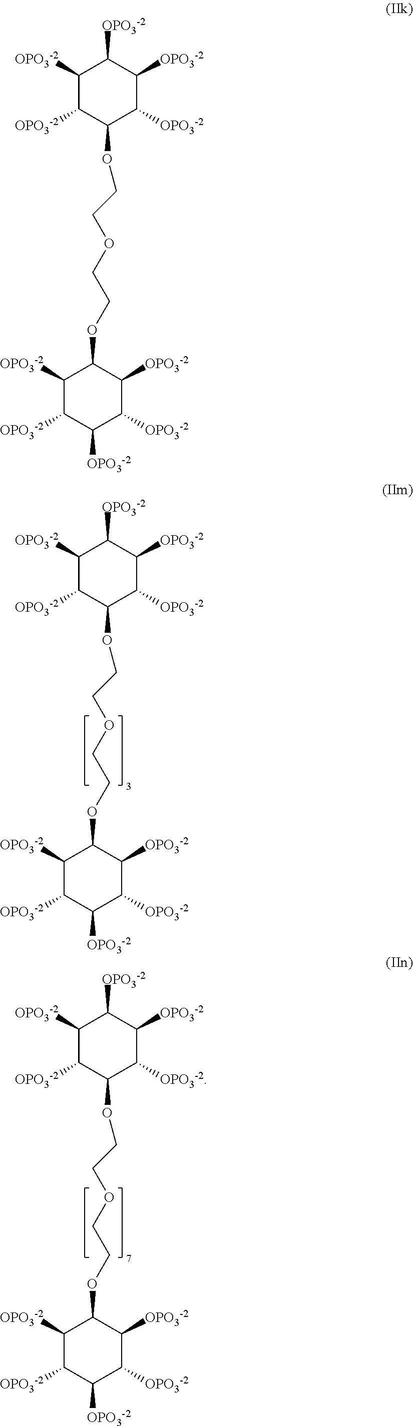

In certain embodiments, the compound of the invention is described by formula (IIg), (IIh), or (IIj)

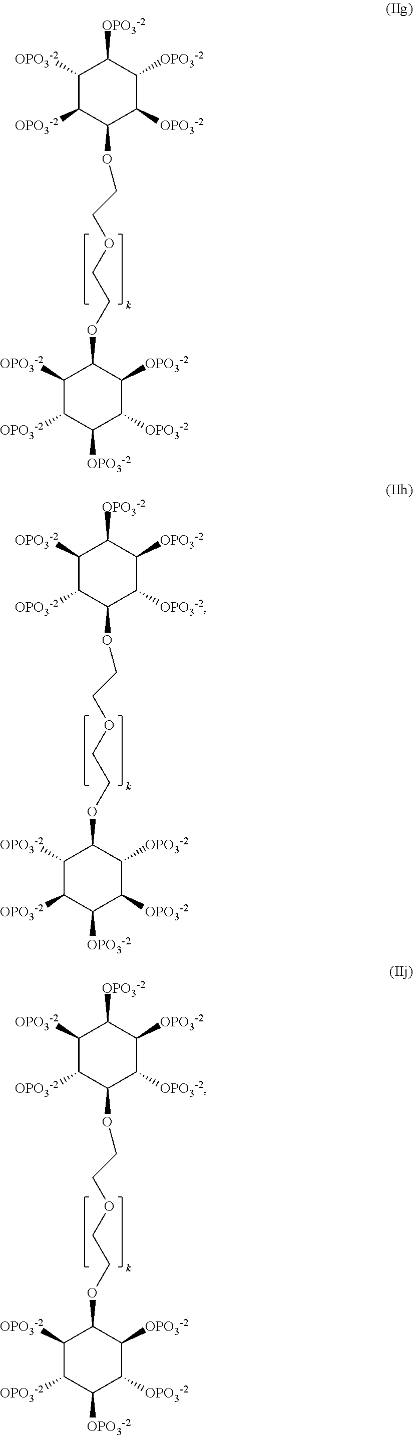

wherein k is an integer selected from 1, 2, 3, 4, 5, 6, 7, 8, 9, 10, 11 and 12.

In certain embodiments, the compound of the invention is described by a general formula (III),

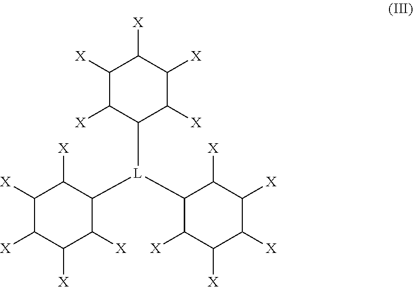

wherein each X independently from any other X can be OPO₃²⁻, OPSO₂²⁻, or OSO₃⁻, and wherein L is a common central linker (also referred to as a “linking moiety” herein) as specified above.

In certain embodiments of the compound described by (III), the linking moiety L is described by a general formula (IIIa)

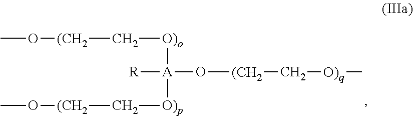

wherein o, p and q independently of each other have a value between 1 and 12, and the sum of o, p and q is ≤30.

In certain embodiments, A is selected from a carbon atom (C), a hydroxy-, amino-, halogen- or carboxy-substituted or an unsubstituted C₄ to C₇ cycloalkyl moiety or a hydroxy-, amino-, halogen- or carboxy-substituted or an unsubstituted aryl. In certain embodiments, A is a carbon atom.

In certain embodiments, R is selected from H and C₁-C₃ unsubstituted or N—, O and/or halogen substituted alkyl. In certain embodiments, is H or unsubstituted alkyl.

In certain particular embodiments of (III), L is described by (IIIa) and o, p and q independently of each other have a value between 2 and 4.

In certain particular embodiments of (III), L is described by (IIIa) and the sum of o, p and q is ≤15.

In certain particular embodiments of (III), L is described by (IIIa) and o, p and q independently of each other have a value between 2 and 4 and the sum of o, p and q is ≤15.

In certain embodiments, R is selected from H and C₁-C₃ unsubstituted or N—, O and/or halogen substituted alkyl. In certain particular embodiments, R is H or unsubstituted alkyl.

In certain embodiments of the compound (III), the linking moiety L is described by a general formula (IIIb)

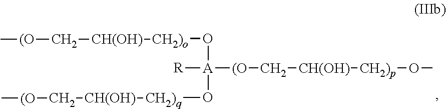

wherein o, p and q independently of each other have a value between 1 and 12, and the sum of o, p and q is ≤30.

In certain embodiments, A is selected from a carbon atom (C), a hydroxy-, amino-, halogen- or carboxy-substituted or an unsubstituted C₄ to C₇ cycloalkyl moiety or a hydroxy-, amino-, halogen- or carboxy-substituted or an unsubstituted aryl. In certain embodiments, A is a carbon atom.

In certain embodiments, R is selected from H and C₁-C₃ unsubstituted or N—, O and/or halogen substituted alkyl. In certain embodiments, is H or unsubstituted alkyl.

In certain particular embodiments of (III), L is described by (IIIb) and o, p and q independently of each other have a value between 2 and 4.

In certain particular embodiments of (III), L is described by (IIIb) and the sum of o, p and q is ≤15.

In certain particular embodiments of (III), L is described by (IIIb) and o, p and q independently of each other have a value between 2 and 4 and the sum of o, p and q is ≤15.

In certain embodiments, of (III), L is described by (IIIa) or (IIIb) and A is a carbon atom. In certain embodiments, of (III), L is described by (IIIa) or (IIIb) and A is selected from a substituted or unsubstituted C₁ to C₄ alkyl. In certain embodiments, of (III), L is described by (IIIa) or (IIIb) and A is selected from a substituted or unsubstituted C₄ to C₇ cycloalkyl. In certain embodiments, of (III), L is described by (IIIa) or (IIIb) and A is selected from a substituted or unsubstituted five- or six-membered aryl.

In certain embodiments of the compound (III), the linking moiety L is described by a general formula (IIIc)

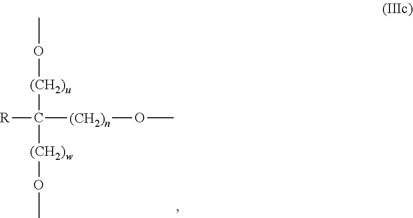

wherein A of formula (IIIb) is a carbon atom (C) and u, v and w independently of each other have a value between 1 and 12, particularly u, v and w independently of each other have a value between 2 and 4; the sum of u, v and w is ≤30, particularly the sum u, v and w is ≤12.

In certain embodiments, R is selected from H and C₁-C₃ unsubstituted or N—, O and/or halogen substituted alkyl, particularly wherein R is H or unsubstituted alkyl.

In certain embodiments, the compound described by general formula (III) has a molecular weight between 1500 to 2500 g/mol.

In certain embodiments, the compound is described by formula (IIId) or (IIIe):

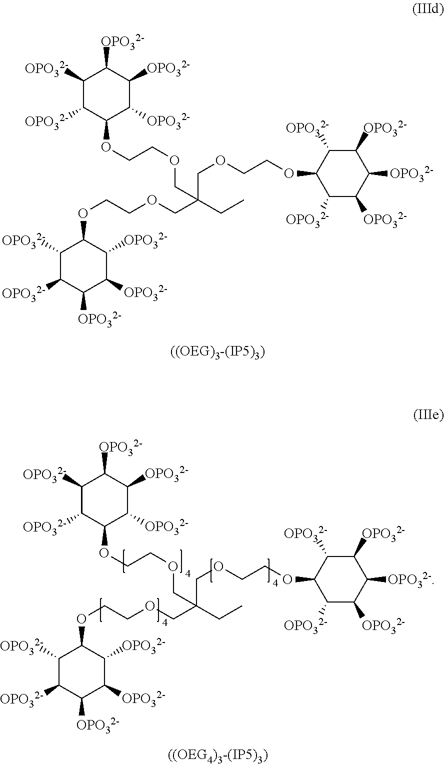

In one embodiment, the compound (IIId) ((OEG)₃-(IP5)₃) has a molecular weight of 1922.21 g/mol. In one embodiment, the compound (IIIe) ((OEG₄)₃-(IP5)₃) has a molecular weight of 2318.69 g/mol.

In certain embodiments, the compound is described by a general formula (IV)

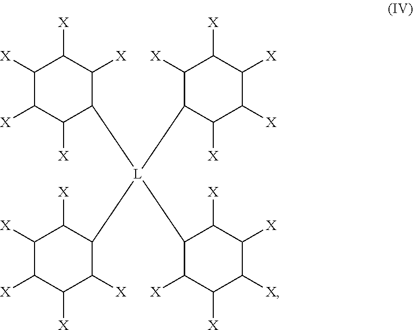

wherein each X independently from any other X can be OPO₃²⁻, OPSO₂²⁻, or OSO₃⁻, and wherein L is a common central linker (also referred to as a “linking moiety” herein) as specified above.

In certain embodiments of the compound described by a general formula (IV), the linking moiety L is described by

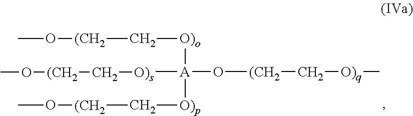

wherein o, p, q and s independently of each other have a value between 1 and 12 and the sum of o, p, q and s is ≤40.

In certain embodiments, A is selected from a carbon atom (C), a hydroxy-, amino-, halogen- or carboxy-substituted or an unsubstituted C₄ to C₇ cycloalkyl moiety or a hydroxy-, amino-, halogen- or carboxy-substituted or an unsubstituted aryl. In certain embodiments, A is a carbon atom.

In certain embodiments, o, p, q and s independently of each other have a value between 2 and 4. In certain particular embodiments, the sum of o, p, q and s is ≤20.

In certain particular embodiments, o, p, q and s independently of each other have a value between 2 and 4 and the sum of o, p, q and s is ≤20.

In certain embodiments of the compound described by (IV), L is (IVa) and A is selected from a substituted or unsubstituted C₁ to C₄ alkyl. In certain embodiments, L is (IVa) and A is selected from a substituted or unsubstituted C₄ to C₇ cycloalkyl. In certain embodiments, L is (IVa) and A is selected from a substituted or unsubstituted five- or six-membered aryl.

In certain embodiments of the compound described by a general formula (IV), the linking moiety L is described by

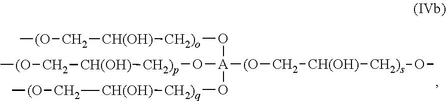

wherein o, p, q and s independently of each other have a value between 1 and 12, particularly o, p, q and s independently of each other have a value between 2 and 4 and the sum of o, p, q and s is ≤40, particularly, wherein the sum of o, p, q and s is ≤20.

In certain embodiments of the compound described by a general formula (IV), the linking moiety L is described by

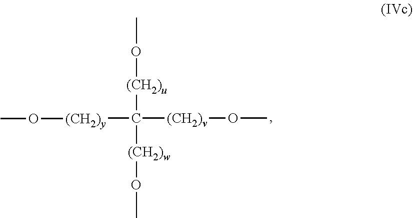

wherein A of formula (IVb) is a carbon atom (C) and u, v, w and y independently of each other have a value between 1 and 12, particularly u, v, w and y independently of each other have a value between 2 and 4 and the sum of u, v, w and y is ≤40, particularly wherein the sum of u, v, w and y is ≤12.

In certain embodiments, the compound described by a general formula (IV) has a molecular weight between 2500 to 3000 g/mol.

In certain embodiments, the compound is described by formula (IVd) or (IVe)

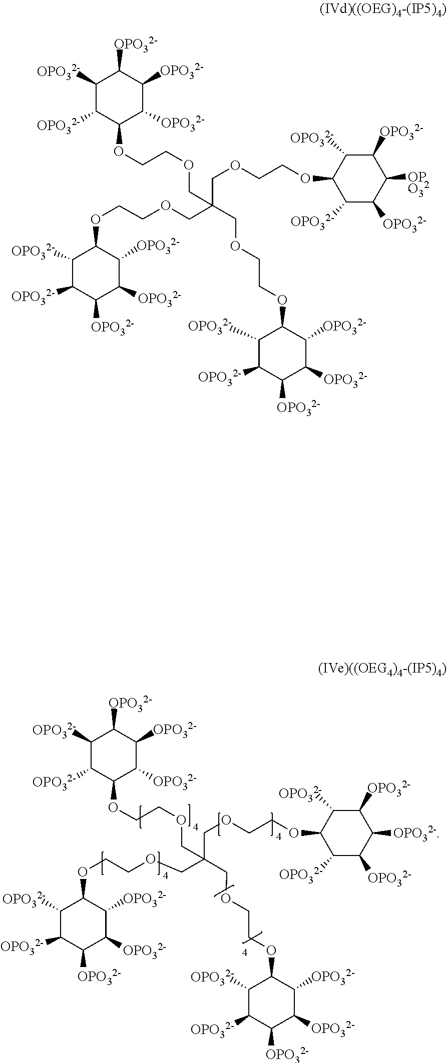

In certain embodiments, the compound of the invention is provided as a sodium salt. In certain embodiments, the compound of the invention is provided as a potassium salt. In certain embodiments, the compound of the invention is provided as a magnesium salt.

The invention further encompasses intermediary products useful in the synthesis of the compounds of the invention, specifically a compound described by general formula Va, Vb or Vc

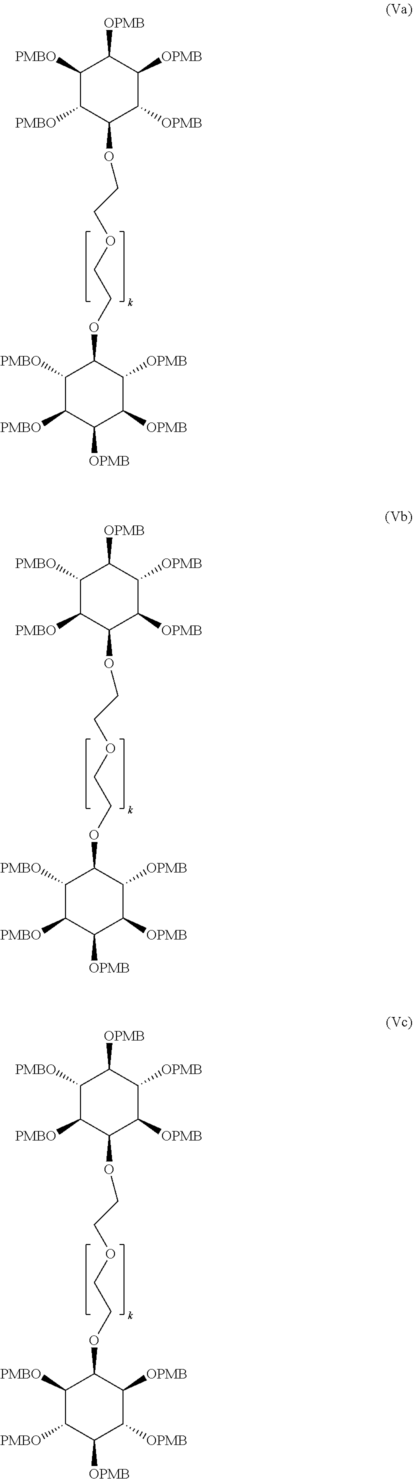

wherein OPMB signifies an O-(bis-paramethoxybenzyl)phosphate moiety and wherein k is an integer selected from 1, 2, 3, 4, 5, 6, 7, 8, 9, 10, 11 and 12.

Another compound useful in the synthesis of the invention is described by the formulae Via, VIb or VIc:

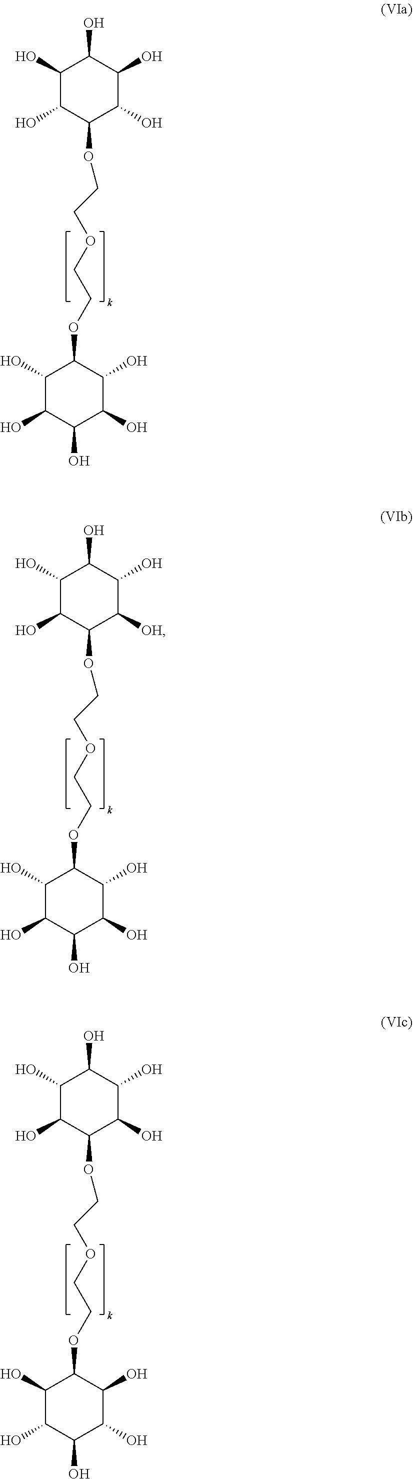

wherein k is an integer selected from 1, 2, 3, 4, 5, 6, 7, 8, 9, 10, 11 and 12.

In one embodiment, the compound (IVd) ((OEG)₄-(IP5)₄) has a molecular weight of 2520.20 g/mol. In one embodiment, the compound (IVe) ((OEG₄)₄-(IP5)₄) has a molecular weight of 3048.84 g/mol.

Another aspect of the invention relates to the use of a compound of the invention as a medicament.

A second aspect of the invention relates to the compound of the invention for use in therapy or prevention of a condition related to pathological crystallization, particularly pathological crystallization of calcium salts and more particularly of calcium oxalate and/or calcium phosphate salts.

In the widest sense, such use of a compound according to the invention encompasses the use of any compound described herein above according to the first aspect of the invention, but also encompasses the structurally slightly broader chemical space of compounds comprising, particularly consisting of, two or more cyclohexanolpentakisester moieties described by a general formula (I) linked by a common central linker L,

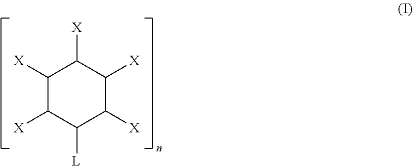

wherein X is selected from OPO₃²⁻, OPSO₂²⁻, OSO₃⁻ or CO₂⁻, n is an integer from 2 to 10, wherein L is a linking moiety comprising or consisting of a linear or branched alkyl, optionally substituted by oxygen, nitrogen, (fluorine), particularly wherein the linking moiety has a molecular weight <1000 g/mol, particularly <700 g/mol, more particularly <500 g/mol, or even <400 g/mol.

In certain embodiments, such compound for use in therapy or prevention of a condition related to pathological crystallization comprises 2, 3, 4, 6 or 8 moieties according to general formula (I). In certain particular embodiments, 2, 3 or 4 such moieties are comprised in the molecule.

In certain embodiments, the compound for use in therapy or prevention of a condition related to pathological crystallization of the invention is described by formula (VII)

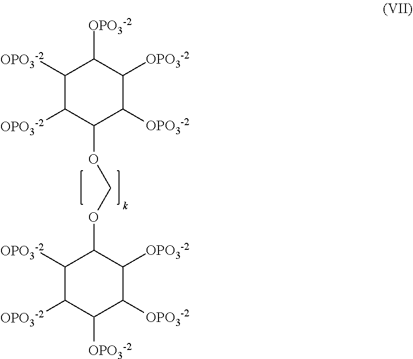

wherein k is an integer from 1 to 20, particularly 1 to 10, more particularly an integer selected from 1, 2, 3, 4, 5, and 6.

Conditions related to pathological calcium crystallization due to pathological calcium oxalate or phosphate crystallization in a patient for which the compounds of the present invention are particularly useful include nephrocalcinosis, nephrolithiasis, calcinosis cutis, kidney stones, cardiovascular mortality (particularly in chronic kidney disease patients), progression of chronic kidney disease and failure of renal transplant grafts. Pathological crystallization has been shown to be associated with morbidity of chronic kidney disease patients, particularly wherein the condition is primary and secondary hyperoxaluria, Dent disease, Bartter's syndrome, distal renal tubule acidosis, neurogenic bladder, autosomal dominant polycystic kidney, calcium oxalate kidney stone formation, struvite stones and renal calcification. Hence the compounds of the present invention are indicated for chronic kidney disease patients in general.

One embodiment of the invention that shows particular high activity is the use of novel divalent IP5 compounds (OEG₄-(IP5)₂, OEG₈-(IP5)₂, OEG₂-(IP5)₂) with similar CaOx inhibitory efficacy (FIG. 2) for the indications listed above. The inventors previously developed a series of small molecules based on inositol hexaphosphate (IP6) analogues (PCT/EP2012/004088, PCT/EP2016/080657) that can decrease the nucleation and growth of CaOx crystals in human urine, and have improved pharmacokinetic (PK) properties. Further, those compounds have been shown to prevent adhesion of CaOx crystals to renal epithelial cells and displayed cyto-protective effects upon CaOx treatment in vitro. While these initial IP6 analogues failed to completely inhibit CaOx crystallization in an in vivo achievable concentration range, the inventors surprisingly found that the potency to inhibit CaOx crystallization was highly dependent on the combination of IP5 moieties. Thus, the inventors developed novel multivalent IP5 compounds by linking two inositol pentaphosphate (IP5) molecules using oligo(ethylene glycol) linkers (OEG), thereby increasing the total negative charge of the molecules. Compared to the previously developed, most potent compound IP5-mono-PEG2 (OEG₂-IP5), the bis-IP5 linked by tris(ethylene glycol) compound OEG₄-(IP5)₂ showed a 2500-fold increase in potency to inhibit CaOx crystallization to below 50% of the control (FIG. 1). This conclusion is drawn from the results of an in vitro assay that measures the propensity for CaOx crystallization of human urine.

The compound may be formulated for intravenous, intraperitoneal, intramuscular, intra-arterial, topical, intravesical or, particularly, subcutaneous administration. Optionally, a pharmaceutically acceptable carrier and/or excipient may be present.

According to another aspect of the invention, a method of treatment or prevention of any of the conditions related to pathological calcium crystallization listed above is provided, comprising the administration of the compound as specified by any of the above formulae to a subject in need thereof.

The skilled person is aware that any compound specifically mentioned herein will be present as a pharmaceutically acceptable salt of the compound. Pharmaceutically acceptable salts comprise the ionized compound and an oppositely charged counterion. Non-limiting examples of pharmaceutically acceptable cationic salt forms include aluminium, benzathine, ethylene diamine, lysine, magnesium, meglumine, potassium, procaine, sodium, tromethamine and zinc.

Pharmaceutical Composition and Administration

Another aspect of the invention relates to a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant and easily handleable product.

The pharmaceutical composition can be formulated for oral administration, parenteral administration, or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions). The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

Wherever alternatives for single separable features are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1 shows the inhibition of CaOx crystallization by IIc (OEG₄-(IP5)₂) and the comparison IP5-mono-PEG2 (OEG₂-IP5) at pH 6.2, and at pH 5.5. CaOx crystallization was induced by adding 1 mM sodium oxalate to humane urine and characterized by light microscopy, followed by an automated image analysis to determine crystal number, hydrate forms (COM—CaOx monohydrate, COD—CaOx dihydrate, n.d.—not defined), and crystal area. Surface area of the different hydrate forms normalized to the total surface area of the control is plotted. IIc/OEG₄-(IP5)₂ led to complete inhibition of COM crystals only at 300 nM, followed by complete CaOx (COM and COD) inhibition at 4 μM. In comparison, for the previous lead compound IP5-mono-PEG₂ COM only inhibition was observed at 11 μM, while complete CaOx (COM and COD) inhibition was not observed in the tested range up to 100 μM.

FIG. 2 shows the inhibition of CaOx crystallization by compounds of the present invention: IIb (OEG₂-(IP5)₂), IIc (OEG₄-(IP5)₂) and IId (OEG₈-(IP5)₂) at pH 6.2.

FIG. 3 shows the inhibition of CaOx crystallization by IP6 analogues previously described in PCT/EP2016/080657 at pH 6.2. CaOx crystallization was induced by adding 1 mM sodium oxalate to humane urine and characterized by light microscopy, followed by an automated image analysis to determine crystal number, hydrate forms (COM—CaOx monohydrate, COD—CaOx dihydrate, n.d.—not defined), and crystal area. Surface area of the different hydrate forms normalized to the total surface area of the control is plotted.

FIG. 4 shows the synthesis of compounds of the invention as described in example 1.

FIG. 5 shows images of CaOx crystal formation inhibition by a compound of the present invention OEG₄-(IP5)₂ (scale bar: 50 μm).

FIG. 6 shows images of CaOx crystal formation inhibition by an IP6 analogue compound OEG₂-IP5 previously described in PCT/EP2016/080657 (scale bar: 50 μm).

FIG. 7 shows the synthesis of compound (OEG₄)₃-(IP5)₃.

FIG. 8 shows changes in the CaOx crystallization pattern induced by IP6 analogues. Effects of (OEG₄)₃-(IP5)₃ on CaOx bulk crystallization in human urine spiked with 1 mM NaOx were assessed by light microscopy at t=7 h. COM—CaOx monohydrate, COD—CaOx dihydrate, n.d.—not defined; N=3, mean total area/field of view+SD for the respective crystal type normalized to the control (without inhibitor).

FIG. 9 shows that OEG₄-(IP5)₂ stabilizes early precursor particles and leads to delayed COD growth. (A) Dose-dependent inhibition of CaOx crystallization by OEG₄-(IP5)₂ in Bis-Tris buffer (pH 6.2) was characterized by SEM at t=7 h. Representative SEM images are shown (scale bar: 1 μm). (B) Crystallization of CaOx with 500 nM OEG₄-(IP5)₂ and without inhibitor was sampled at t=10 min and imaged by SEM (scale bar: 1 μm). Crystallinity was characterized by TEM with SAED (inlets).

FIG. 10 shows inhibition of CaOx adhesion to renal epithelial cells by IP6 analogues. Anti-adhesive effects of (A) OEG₄-(IP5)₂, (B) OEG₂-IP5 and (C) (OEG₂)₂-IP4 on CaOx attachment to RPTEC/TERT1 cells in vitro were assessed by differential interference contrast microscopy followed by quantification of the crystal occupied area. Cells at confluence were treated with 150 μg/cm² CaOx premixed with compound for 30 minutes, before washing, fixation and imaging. (D) Example images of CaOx premixed with/without OEG₄-(IP5)₂ adhering to the confluent cell layer (Leica CTR6000, 63× oil objective). (E) To assess the capacity of OEG₄-(IP5)₂ to remove already bound CaOx of cell layers an adapted version of the assay described above was performed. RPTEC/TERT1 cells were incubated with CaOx for 30 minutes, unbound CaOx was removed by washing with PBS and cells were further incubated with OEG₄-(IP5)₂ or medium control for 2 h. (N=3, mean+SD normalized to the CaOx ctrl, one-way ANOVA with Dunnett's multiple comparison, **** p<0.0001, *** p<0.001, ** p<0.01, * p<0.05, compared to CaOx control.)

FIG. 11 shows that CaOx induced cellular injury of renal epithelial cells is prevented by OEG₄-(IP5)₂ in vitro. (A) Heatmap and hierarchical clustering of relative gene expression levels determined by RNA sequencing (differentially expressed genes between CaOx versus CaOx-OEG₄-(IP5)₂ with p<0.01, log 2 ratio=0.5, number of significant features 2000). (B) Gene set enrichment analysis of gene expression levels in the CaOx relative to the medium group and corresponding values in the CaOx-OEG₄-(IP5)₂ group. Top gene ontology terms and corresponding normalized enrichment score for differentially up- or down-regulated genes in the CaOx group relative to the medium control group are plotted (top 10 upregulated gene ontology terms, all FDR≤0.05 and downregulated gene ontology terms with FDR≤0.05 are shown). Corresponding values for differentially expressed genes in the CaOx-OEG₄-(IP5)₂ group are given (FDR≤0.05 is indicated by dark blue, FDR>0.05 by light blue). (C) Gene expression levels of genes involved in inflammatory and immune response, cellular signalling and ECM production in the different treatment groups. (D) Dose-dependent decrease in the number of dead cells by pre-incubation of CaOx with OEG₄-(IP5)₂ before treatment of RPTEC cells in comparison to CaOx only treated cells was assessed by a viability stain (mean+SD normalized to CaOx ctrl, one-way ANOVA with Dunnett's multiple comparison, **** p<0.0001, ** p<0.01). (E) Example brightfield images showing CaOx deposition (black spots) and red fluorescence images indicating cell death of cells receiving no treatment, CaOx only or CaOx+10 μM OEG₄-(IP5)₂ treatment (Leica CTR6000, 10× objective, scale bar: 100 μm). (N=3 for all experiments.)

Table 1 shows data from a screening of calcification inhibitors for activity in inhibiting growth of CaOx crystals. IP6 analogues previously described in PCT/EP2016/080657 and compounds OEG₂-(IP5)₂, OEG₄-(IP5)₂, OEG₈-(IP5)₂ and (OEG₄)₃-(IP5)₃ of the present invention are listed.

### EXAMPLES

### Example 1: Synthesis of IIc (OEG₄-(IP5)₂)

Step 1:

Reagents: DMF, sodium hydride, p-methoxybenzyl chloride.

Myo-inositol-1,3,5-orthoformate is dissolved in dry DMF under nitrogen atmosphere, cooled to 0-5° C. and added portion-wise with sodium hydride (2.2 eq.). After stirring 1 h at room temperature, p-methoxybenzyl chloride (2.3 eq.) is added dropwise. The mixture is stirred at room temperature for 12 h, then it is added with water and extracted with ethyl acetate. Solvent removed under vacuum and residue crystallized with diethyl ether. Yield 70%.

Step 2:

Reagents: MeOH; HCl 2N.

The product of step 1 (1) is dissolved in 10 vol of methanol, added with 1 vol of HCl 2 N and heated at 50° C. for 3 hs. After cooling, the mixture is neutralized with ammonium hydroxide 30% and left overnight at room temperature. The solid formed, is filtered, washed with water and dried. After crystallization with diethyl ether compound 2 is obtained as whitish solid. Yield 80%.

Step 3:

Reagents: DMF, sodium hydride, p-methoxybenzyl chloride.

The product of step 2 (2) is dissolved in dry DMF under nitrogen atmosphere, cooled at 0-5° C. and added with sodium hydride (2 eq.) portion-wise. After 1 h at room temperature, the mixture is cooled at 0-5° C. and added with p-methoxybenzyl chloride (2 eq.). After 12 h at room temperature, the mixture is added with water and extracted with ethyl acetate. The residue is purified by column chromatography eluting with hexanes/ethyl acetate 6/4, then crystallized with diethyl ether. Yield 35%.

Step 4:

Reagents: dichloromethane, Tosyl chloride, triethylamine.

Tetraethylene glycol is dissolved in dry dichloromethane, added with triethylamine (3 eq.) and then with p-toluenesulfonyl chloride (2.5 eq.). After stirring for 12 h at room temperature, the solvent is removed under vacuum and the residue purified by column chromatography, eluting with ethyl acetate/hexanes 7/3. Yield 80%.

Step 5:

Reagents: DMF, sodium hydride, tetraethylene glycol bis tosylate (step 4).

The product of step 3 (3) is dissolved in dry DMF under nitrogen atmosphere, cooled to 0-5° C. and added with sodium hydride (1.1 eq.) portionwise. After stirring 1 h at room temperature, the mixture is again cooled to 0-5° C. and added with a solution of compound 4 (0.5 eq.) in DMF (3 vol). After stirring for 12 h at room temperature, the mixture is added with water and extracted with ethyl acetate. After removal of the solvent the mixture is purified by chromatography eluting with hexanes/ethyl acetate.

Yield 40%.

Step 6:

Reagents: Dichloromethane, trifluoroacetic acid. Not purified.

The product of step 5 (5) is dissolved in dichloromethane (10 vol) and added with trifluoroacetic acid (2 vol). After stirring for 3 h at room temperature, the solvent is removed under vacuum and the residue used for the next step without further purification. Yield quantitative.

Step 7:

Reagents: DMF, 5-methyl-tetrazole, dibenzyloxy-diisopropylphosphoroamide, then H₂O₂.

The product of step 6 (6) is dissolved in dry DMF (10 vol) under nitrogen atmosphere, added with 5-methyltetrazole (9 eq.) and cooled to −10° C., followed by drop-wise addition of dibenzyloxy-diisopropylphosphoroamide (6 eq.). After stirring for 12 h at room temperature, the mixture is cooled to 0-5° C. and added drop-wise with H₂O₂ 30% (9 eq.). After stirring 1 h at room temperature, water is added and the mixture is extracted with ethyl acetate. After removal of the solvent, the residue is purified by column chromatography eluting with chloroform/methanol 96/4. Yield 40%.

Step 8:

Reagents: H₂, Pd/C. Magnesium salt formation with Magnesium ethoxide.

The product of step 7 (7) is dissolved in methanol (15 vol), added with Pd/C 10% and hydrogenated at room temperature and room pressure for 24 hs. After filtration of the catalyst, the solvent is removed under reduced pressure and compound 8 as acid is obtained. Yield quantitative. The formation of the magnesium salt is performed by adding an aqueous solution of magnesium ethoxide to the aqueous solution of compound 8, followed by distillation of the solvent under vacuum.

### Example 2: Synthesis of (OEG₄)₄-(IP5)₄

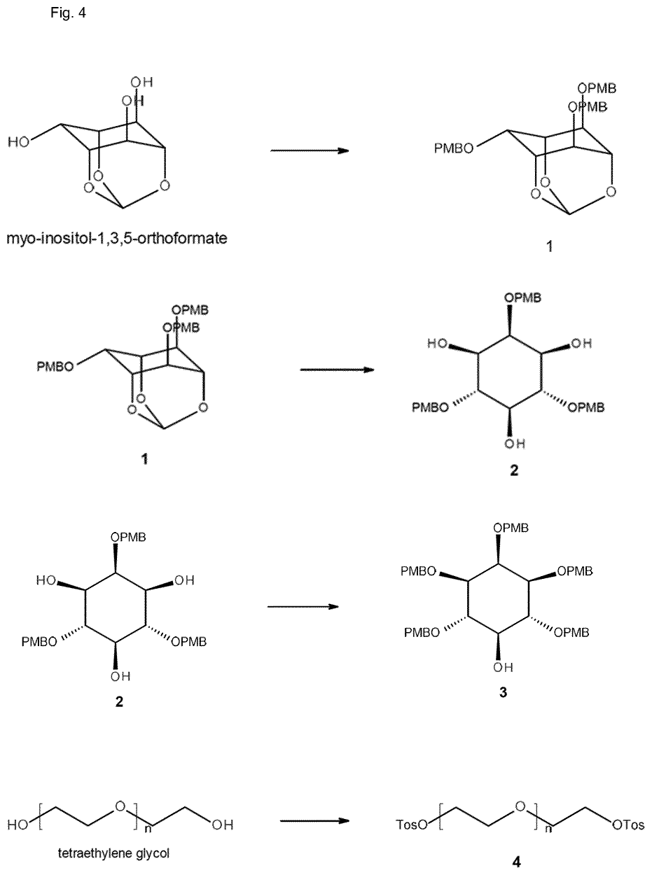

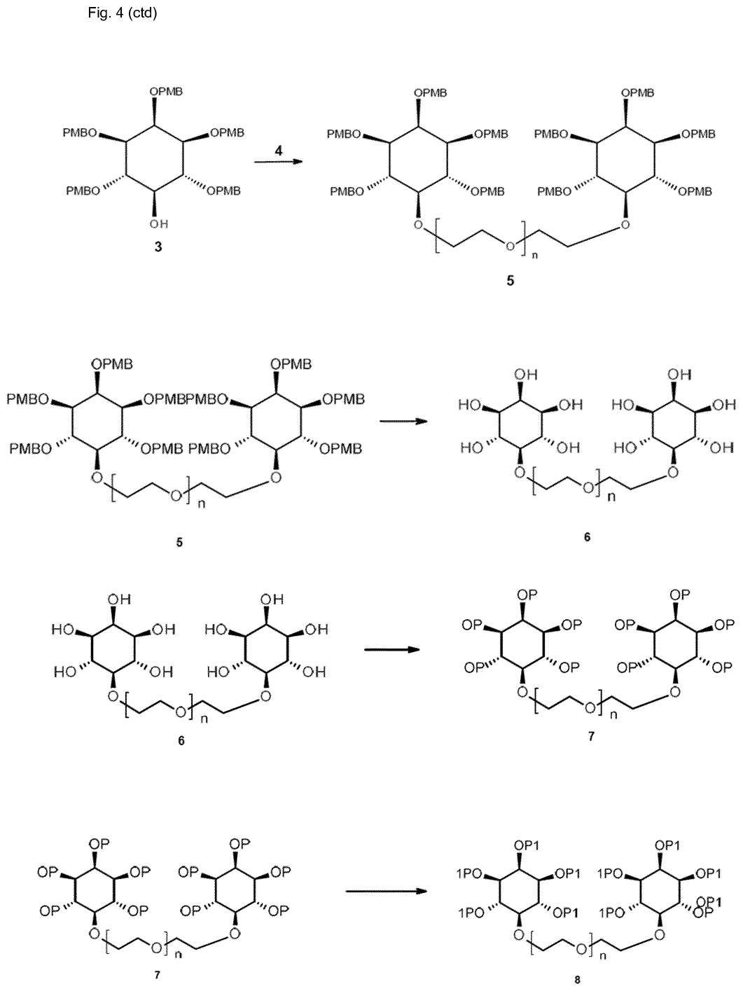

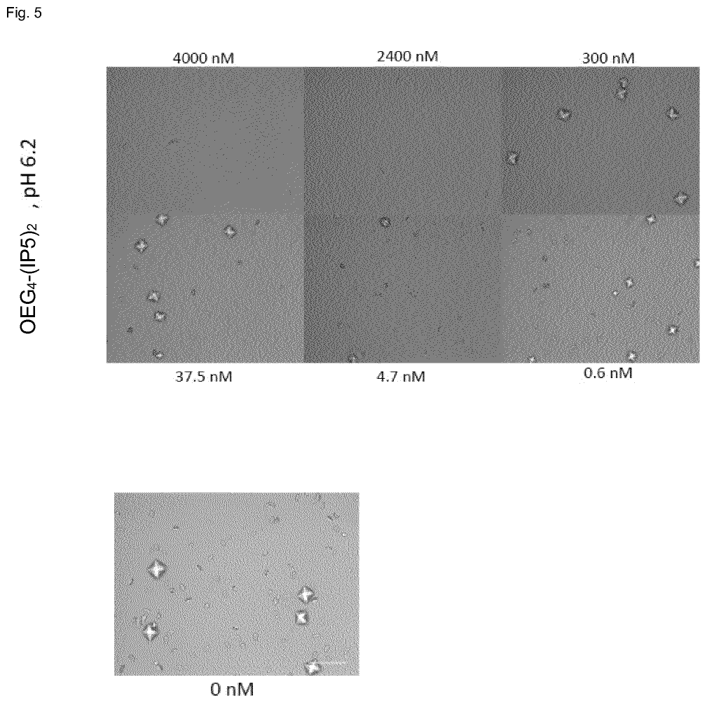

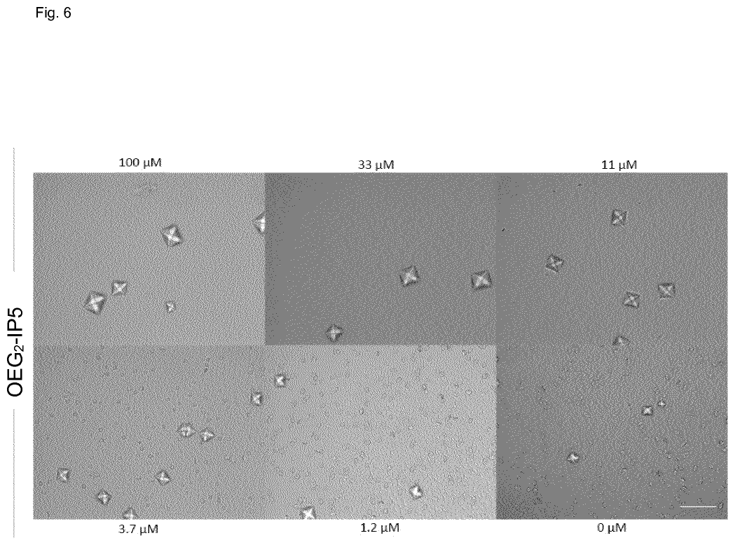

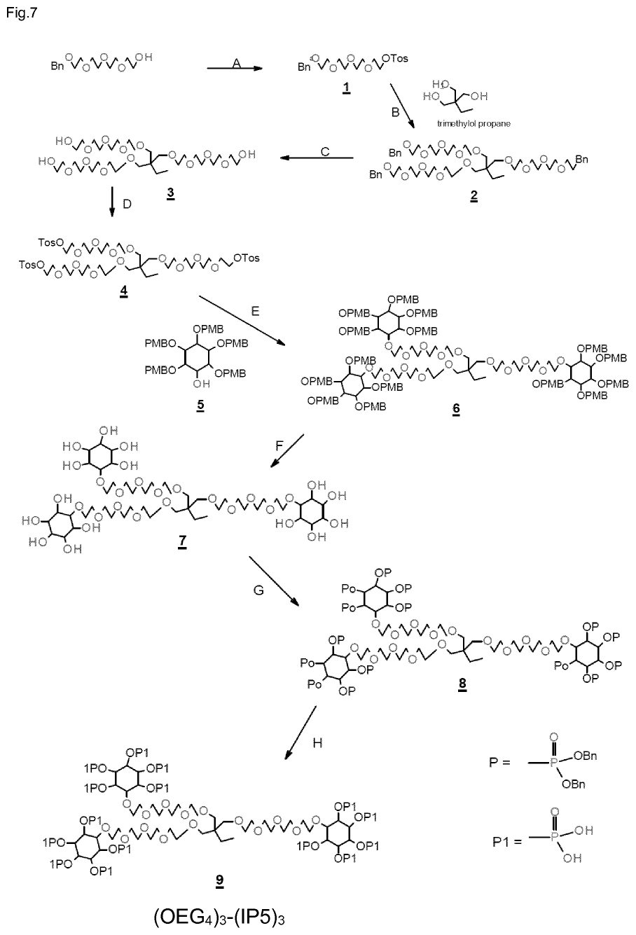

Compound 5 (PMB=p-methoxybenzyl; FIG. 7) was prepared in three steps starting from myoinositol-1,3,5-orthoformate, as described in the synthesis of OEG₄-(IP5)₂ (Example 1; FIG. 4).

Reagents of step A (FIG. 7): P-Toluenesulfonyl chloride; dichloromethane, triethyl amine. Column chromatography. Yield 80%.

Reagents of step B (FIG. 7): Trimethylol propane, DMF, sodium hydride. Column chromatography. Yield 45%.

Reagents of step C (FIG. 7): Methanol, H₂, Pd/C. Yield 80%.

Reagents of step D (FIG. 7): P-Toluenesulfonyl chloride; dichloromethane, triethyl amine. Column chromatography. Yield 70%.

Reagents of step E (FIG. 7): Compound 5 as shown in FIG. 7, DMF, sodium hydride. Column chromatography. Yield 20%.

Reagents of step F (FIG. 7): Dichloromethane, trifluoroacetic acid. Yield quantitative.

Reagents of step G (FIG. 7): DMF, dibenzyl-N,N-diisopropylphosphoramidite, 5-methyltetrazole. Column chromatography. Yield 30%.

Reagents of step H (FIG. 7): Methanol, H₂, Pd/C. Yield 90%. Then sodium salt formation, quantitative.

### Example 3: Inhibition of CaOx Crystallization

For comparison of the compounds' efficacy to inhibit calcium oxalate (CaOx) crystallization a simple in vitro assay in urine was used. In brief, human urine (pH 6.2) was mixed with compound, followed by the addition of 1 mM sodium oxalate inducing CaOx crystallization. After 7 hours of incubation at room temperature crystallization was measured by light microscopy, employing an automated image analysis to detect, classify and quantify the CaOx crystal types formed, namely CaOx monohydrate (COM) and CaOx dihydrate (COD).

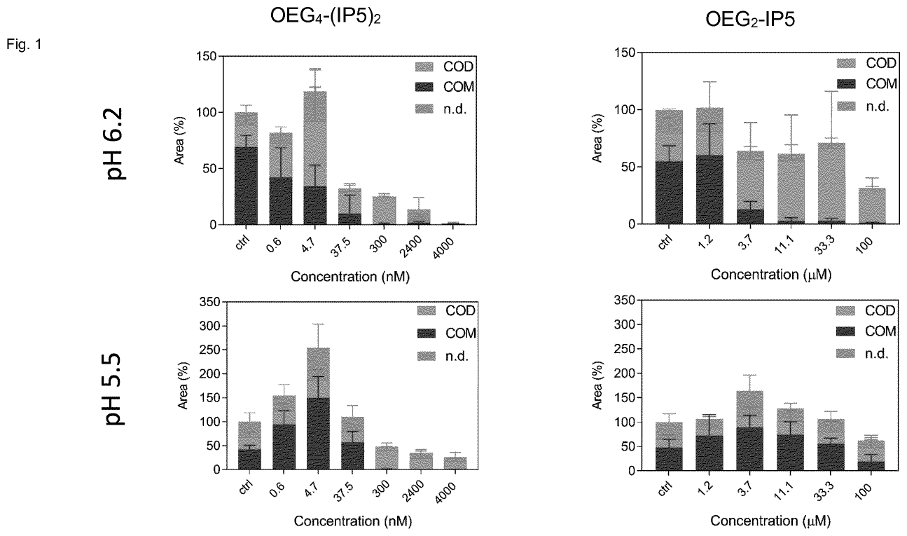

The inventors found that all three novel divalent IP5 structures showed similar CaOx inhibitory efficacy in vitro with complete inhibition of CaOx crystallization at 4 μmol/L (FIG. 1). The length of the linker did not have an important impact on inhibitory efficacy. Compared to the previously developed, most potent compound IP5-mono-PEG2 (OEG₂-IP5) (PCT/EP2012/004088 & PCT/EP2016/080657), OEG₄-(IP5)₂ showed a 2500-fold increase in potency to inhibit CaOx crystallization to below 50% of the control (FIG. 1).

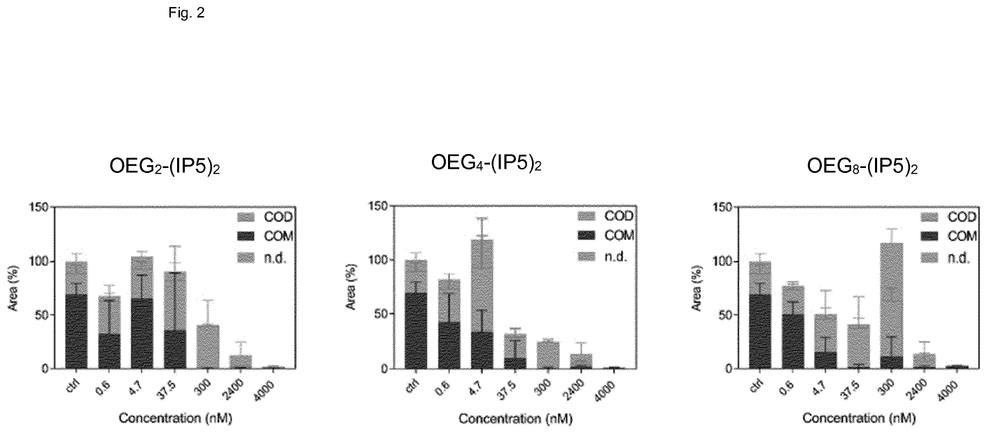

The inventors observed that first, the compounds at sub-inhibitory concentrations lead to a shift in crystallization from COM towards COD, further increasing concentrations of compound can then completely inhibit CaOx growth with an unexpected efficiency. All tested divalent IP5 structures showed complete CaOx inhibition in the in vitro assay at low micromolar concentrations, which are expected to be achieved in vivo (FIG. 2, Table 1).

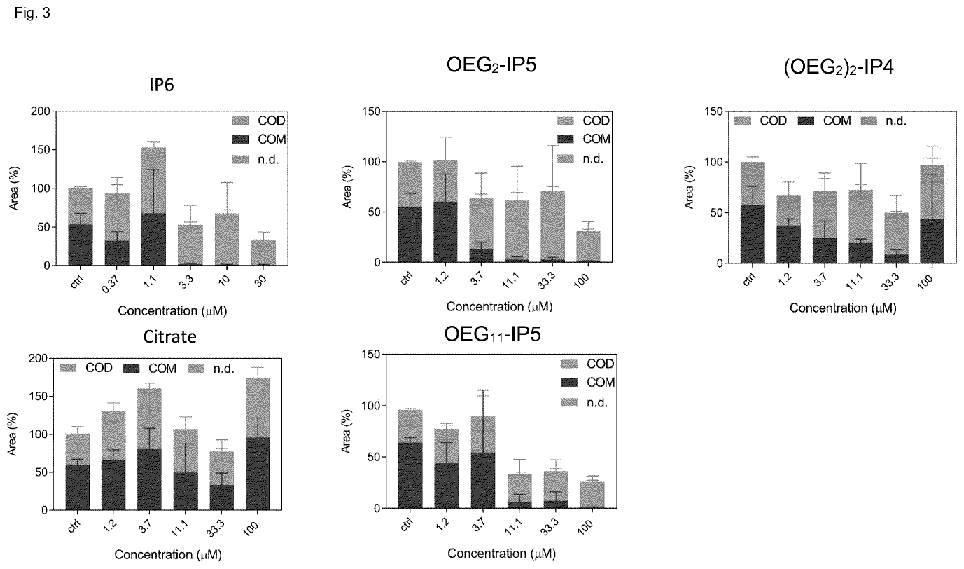

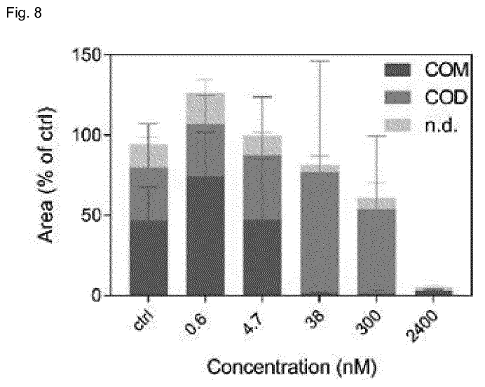

The inventors showed that the efficacy to inhibit CaOx crystallization is dependent on the number of phosphate groups on the molecule and that complete inhibition of crystallization was observed with all OEGₓ-(IP5)₂ compounds at 4 μmol/L (Table 1). Furthermore, IP6 and other compounds tested up to 30 μM or 100 μM, respectively, did not lead to complete CaOx inhibition (FIG. 3, Table 1). Minimal impact of the length of the OEG chain or length of linker on total CaOx crystallization, was observed mostly affecting COM-COD formation dynamics. Moreover, decreasing the pH to 5.5 reduces the activity of OEG₂-IP5 more drastically than of OEG₄-(IP5)₂. The compound OEG₂-IP5 at 100 μM did not result in <50% complete inhibition or COM inhibition. In addition, the compound OEG₄-(IP5)₂ retained COM inhibition at 0.3 μM and 50% complete inhibition at 2.4 μM (FIG. 1, Table 1). The compound (OEG₄)₃-(IP5)₃ displayed COM inhibition at 0.04 μM and complete inhibition at 2.4 μM (FIG. 8, Table 1).

TABLE 1

| | | Complete | 50% complete | COM |
|---|---|---|---|---|
| | | inhibition | inhibition | inhibition |
| | | (i.e <5% total | (<50% total | (<5% of total |
| | Compound | area) (μM) | area) (μM) | area) (μM) |
| pH 6.2 | IP6 | >30 | 30 | 3 |
| | OEG₂-IP5 | >100 | 100 | 11 |
| | OEG₁₁-IP5 | >100 | 11 | 100 |
| | (OEG₂)₂-IP4 | >100 | >100 | >100 |
| | OEG₂-(IP5)₂ | 4 | 0.3 | 0.3 |
| | OEG₄-(IP5)₂ | 4 | 0.04 | 0.3 |
| | OEG₈-(IP5)₂ | 4 | 2.4 | 2.4* |
| | (OEG₄)₃-(IP5)₃ | 2.4 | 2.4 | 0.04 |
| | Citrate | >100 | >100 | >100 |
| pH 5.5 | OEG₂-IP5 | >100 | >100 | >100 |
| | OEG₄-(IP5)₂ | >4 | 2.4 | 0.3 |

*High COD crystallization - possible misclassification as COM at 300 nM

### Example 4: OEG₄-(IP5)₂ Inhibits CaOx Crystallization by Early-Stage Precursor Stabilization

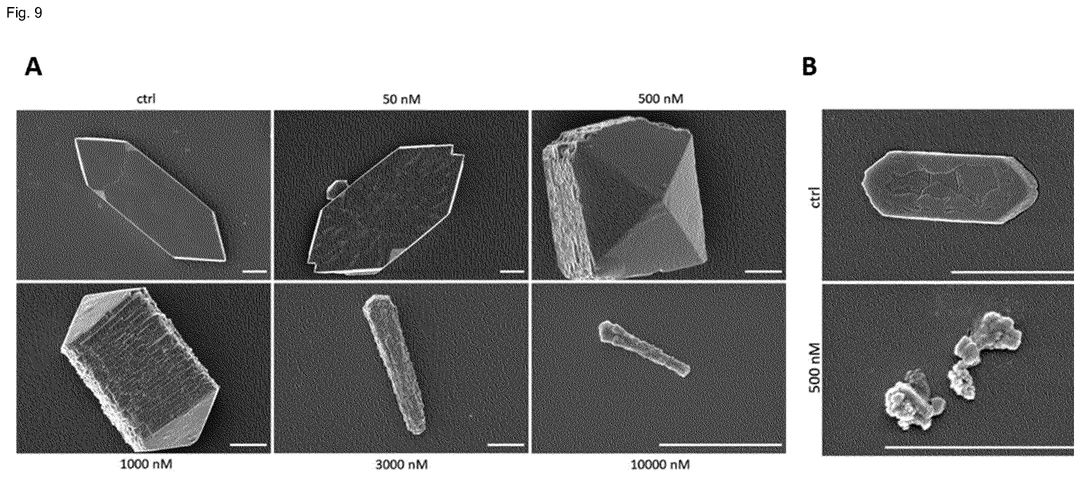

Scanning electron microscopy (SEM) experiments of early stage crystallization at t=10 min, revealed the presence of both, COM shaped- and round nanosized particles, while with 500 nM OEG₄-(IP5)₂ only nanosized particles were detected (FIG. 9B). Nanosized particles were still present at t=1 h with 0.5-10 μM of inhibitor, in contrast to micrometer sized COM crystals being predominant in the control sample, suggesting the stabilization of early stage, nanosized CaOx particles by OEG₄-(IP5)₂.

SEM experiments further confirmed a dose-dependent shift from predominately COM towards COD crystallization, and a COD face-specific growth inhibition with OEG₄-(IP5)₂ (FIG. 9A). Taken together these results indicate that OEG₄-(IP5)₂ alters the crystallization process at several stages, namely crystal nucleation and growth kinetics, as well as crystal polymorphism and shape.

### Example 5: OEG₄-(IP5)₂ Blocks CaOx Adhesion to Renal Epithelial Cells In Vitro More Efficiently than OEG₂-4P5

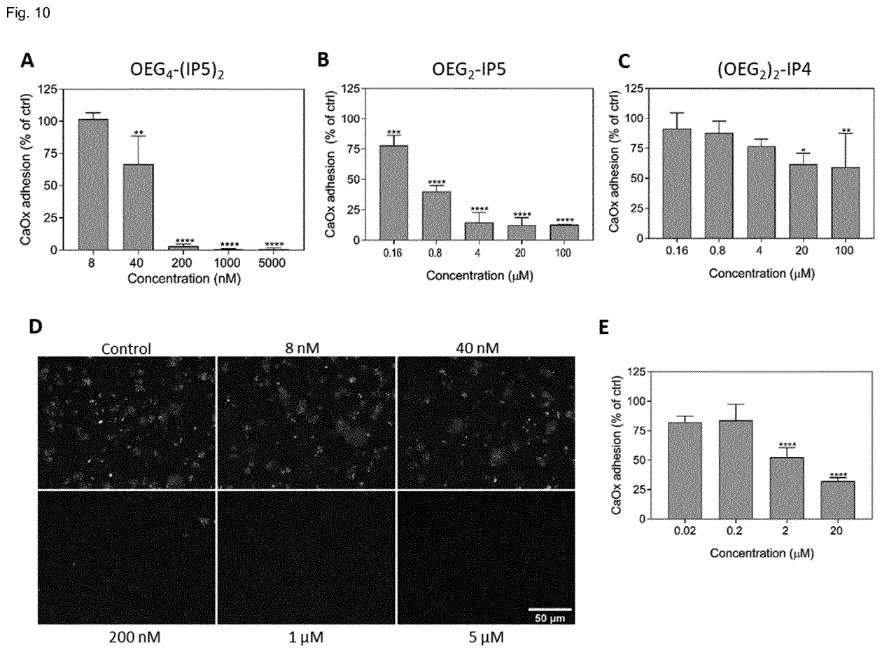

Anti-adhesive effects of chosen IP6 analogues on CaOx crystals to renal proximal tubular epithelial cells (RPTECs) in vitro were investigated by differential interference contrast microscopy. Comparison of OEG₄-(IP5)₂, OEG₂-IP5 and (OEG₂)₂-IP4 showed that concentrations of 200 nM, 4 μM and above 100 μM, respectively, lead to <25% of CaOx adhesion compared to the control (FIG. 10A-D). Citrate treatment did not lead to any detectable protection against CaOx adhesion up to 100 μM. Additionally, OEG₄-(IP5)₂ not only inhibited CaOx adhesion, but also reversed the binding of prebound CaOx crystals at low micromolar concentrations (FIG. 10E).

### Example 6: Cyto-Protective Effects of OEG₄-(IP5)₂ on CaOx Induced Cellular Injury

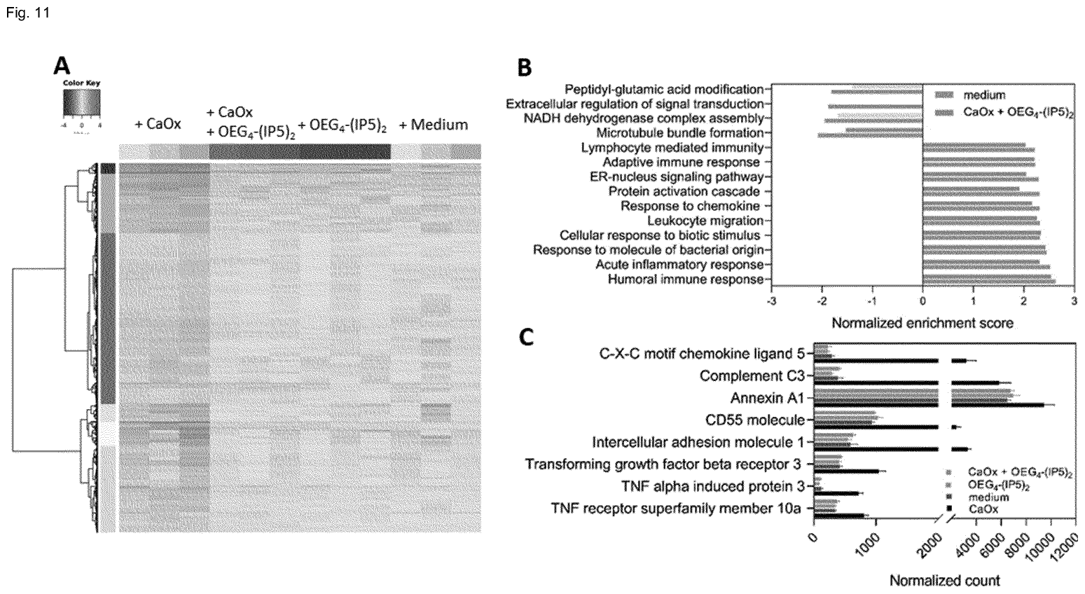

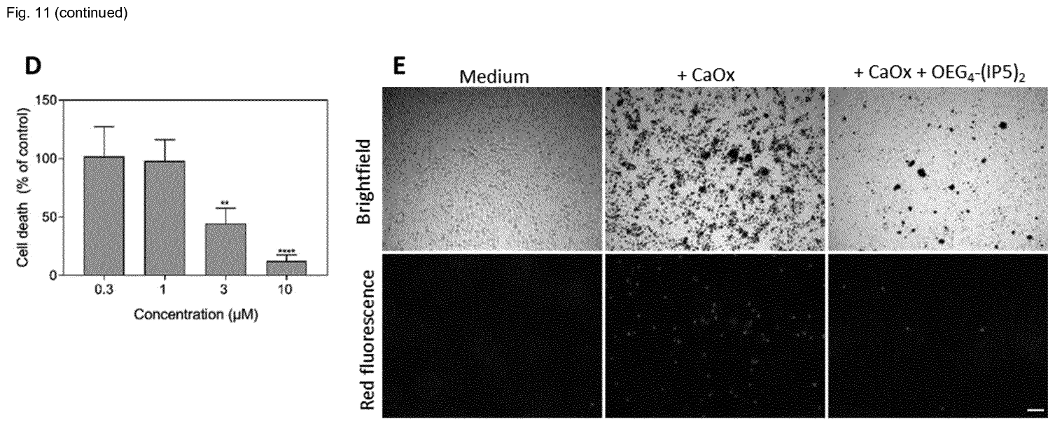

RNA sequencing of RPTECs showed a similar gene expression profile between medium control, OEG₄-(IP5)₂ control and cells treated with CaOx premixed with OEG₄-(IP5)₂, while CaOx treatment alone induced drastic changes (FIG. 11A). Gene set enrichment analysis of differently expressed genes in the CaOx over medium samples revealed alteration of genes mainly involved in immune and inflammatory responses, which coincides with literature, as well as structural changes (e.g. microtubule organization) and protein modification and signaling effects (e.g. peptidyl-glutamic acid modification, ER-nucleus signaling pathway) (FIG. 11B). Comparing the top 10 enriched gene sets of differentially expressed genes in CaOx over medium samples versus CaOx over CaOx-OEG₄-(IP5)₂ samples showed similar changes, indicating that premixing OEG₄-(IP5)₂ with CaOx can prevent CaOx induced transcriptomic changes (FIG. 11B). Elevated expression of TNF alpha and TGF beta signaling pathway genes, cell surface glycoprotein, C-X-C motif chemokine ligand and complement cascade genes, in the CaOx group compared to all other treatment groups were detected (FIG. 11C). Further, CaOx crystal treatment triggered plasma membrane damage, as evidenced by ethidium homodimer-1 staining, which was prevented by OEG₄-(IP5)₂ in a dose-dependent manner (FIG. 11 D,E).

Materials and Methods

Materials

Compounds were custom synthesized by Chimete Srl (Tortona, Italy). Phytic acid dodecasodium salt was purchased from Biosynth AG (Thal, Switzerland). Pooled human urine was purchased from Lee Biosolutions (Maryland Heights, Mo.). Oxalate assay kit (MAK315), calcium colorimetric assay kit (MAK022), Bis-Tris, sodium oxalate, formalin solution neutral buffered 10% and Mowiol 4-88 were purchased from Sigma-Aldrich (St. Louis, Mo.). Sodium chloride and calcium chloride dehydrate were obtained from Merck (Kenilworth, N.J.). Calcium oxalate monohydrate was purchased from abcr (Karlsruhe, Germany). 8-well glass bottom slides (80827) were purchased from ibidi (Martinsried, Germany). Trisodium citrate dihydrate analytical grade, Nunc Lab-Tek II chamber slides, cell culture plates, Live/Dead Viability/Cytotoxicity kit for mammalian cells and phosphate buffered saline (PBS) were purchased from Thermo Fisher Scientific (Rochester, N.Y.). RPTEC/TERT1 cells, ProxUp basal medium and supplements were obtained from Evercyte (Vienna, Austria). RNeasy kit was purchased from Quiagen (Hilden, Germany) and TrueSeq RNA kit from Illumina (San Diego, Calif.).

CaOx Screening Assay and Analysis

Human urine was stored at −20° C. in 50 mL aliquots. After thawing, aliquots were centrifuged, filtered using a 0.45-μm syringe filter and pH set to 6.2. Oxalate concentrations of human urine samples were determined using the Oxalate Assay kit following the kit protocol, and found to be <100 μM for the samples used. Dilutions of sodium and compounds were prepared in Bis Tris buffer (50 mM, 150 mM NaCl, pH 6.2). Urine was mixed with compound in Eppendorf tubes, and subsequently sodium oxalate was added. The final assay mixture of 1 mL total volume contained 90% urine, 5% sodium oxalate (1 mM) and 5% compound dilution. Immediately upon sodium oxalate addition, the final assay mixture was vortexed and added to the imaging slide (8 well glass bottom chamber, ibidi). 400 μL per well were added and two wells for each sample were prepared. Crystallization was assessed after 7 h incubation at room temperature, using a Leica CTR6000 microscope (Leica Microsystems, Wetzlar, Germany) in brightfield mode. For visualization, images with a 40× objective were used. For quantification 2 wells/sample with five 20× objective images each were used. Without addition of sodium oxalate no crystallization was observed.

Inhibition of seeded crystals was evaluated in a similar manner. Crystallization was induced with 1 mM sodium oxalate in urine as above followed by compound addition after 1.5 h incubation at room temperature directly to each well. Samples were imaged before compound addition at t=1.5 h and afterwards at t=7 h total incubation time.

Quantification and classification of crystals was performed in Matlab (version R2016b or R2018b). In brief, crystals were segmented using edge detection and watershed algorithms and subsequently shape, intensity and texture features for each crystal were extracted. The extracted features served as input for a semi-supervised classification approach to distinguish COM (class 0), COD (class 1), not determined structures (class 2) and background noise (class 3). A cubic support vector machine (SVM) classifier was trained and subsequently used for classification of testing images.

All testing images were processed using the Batch Processing App. Efficacy of compounds to inhibit CaOx crystallization was compared by analysing the total area per field of view for each crystal type. The sum of the total area of the three crystal classes—COM, COD and unclassified, was normalized to the control within each experiment (N=3). Due to the inherent variability of the crystallization process itself, normalization within each experiment decreased the effect of inter-experimental variability.

Scanning Electron Microscopy

CaOx crystals for SEM were prepared in Bis Tris buffer (50 mM, 150 mM NaCl, pH 6.2) containing 1 mM NaOx, 2 mM CaCl₂ and the indicated concentration of compound. 12 mm round glass coverslips were placed at the bottom of 24 well plates. First, stock solutions of 20× final concentrations of CaCl₂ and inhibitor, and 10× final concentration of NaOx were prepared in the Bis Tris buffer. Assay mixture was prepared in Eppendorf tubes by first adding 800 μL of Bis Tris buffer, followed by addition of 50 μL CaCl₂ (20× concentration) and 50 μL of inhibitor (20× concentration) and vortexing. Then 100 μL NaOx (10× final concentration) was added, the assay mixture was vortexed and immediately added to the prepared 24 well plate. 400 μL per well and 2 wells per sample were prepared. Samples were incubated at room temperature for the indicated time. Example images were taken using a Leica CTR6000 microscope (Leica Microsystems) in brightfield mode before samples were washed once with double distilled water (ddH₂O) and drying at room temperature. Samples were imaged by brightfield microscopy again after drying to confirm little effects of the drying process on crystal morphology. After drying, glass coverslips were mounted on SEM stubs with silver paint and coated with a 6 nm layer of Platinum/Palladium using a CCU-010 Metal Sputter Coater (Safematic, Bad Ragaz, Switzerland). Samples were imaged using a Magellan 400 FEI SEM microscope (ThermoFisher Scientific, Rochester, N.Y.) in the secondary electron mode using the TLD detector.

Cell Experiments

RPTEC/TERT1 human proximal tubule cells (Evercyte) were cultured in ProxUp basal medium (Evercyte, DMEM/Ham's F12, Hepes buffer and GlutaMAX™) mixed with ProxUp supplements (Evercyte) at 37° C. at 5% CO₂ according to the manufacturer's recommendations. Cells were regularly tested for Mycoplasma infection.

Cell viability was assessed using the Live/Dead Viability/Cytotoxicity kit (ThermoFisher Scientific). Cells were cultured in 24 well plates and 48 h after seeding (upon reaching confluence) treated with 150 μg/cm² Calcium oxalate monohydrate (abcr) in ProxUp basal premixed with compound. After 48 h treatment, cell viability was determined by staining with the Live/Dead cytotoxicity kit (ThermoFisher Scientific), according to the kit protocol. Images were obtained by epifluorescence microscopy with a Leica CTR6000 microscope (Leica Microsystems, 3 wells/sample, 3 images/well) and the number of red fluorescence dots was quantified.

For quantification of CaOx adhesion to RPTEC cells, cells were cultured on 8-well Nunc Lab Tek chamber slides (ThermoFisher Scientific), and upon confluence at 48 h treated with COM (abcr) premixed with compound in ProxUp basal medium (pH set to 6.9) for 30 min at 37° C., 5% CO₂. Cells were washed twice with phosphate buffered saline (PBS, pH 7.4), fixed with 10% neutral buffered formalin for 15 min, and rinsed with PBS, followed by a final rinse with ddH₂O. The chamber of the slide was removed and cells mounted with Mowiol. Images were obtained with a Leica CTR6000 microscope (Leica Microsystems) in DIC mode, 63× oil objective (3 wells per sample; 8 images per well) and crystal occupied area quantified.

RNA Sequencing

Cells were cultured as described in the cell viability assay. Four treatment groups consisted of a medium control (ProxUp basal), 150 μg/cm² Calcium oxalate monohydrate (abcr) only, 150 μg/cm² Calcium oxalate monohydrate (abcr) premixed with 10 μM OEG₄-(IP5)₂ and 10 μM OEG₄-(IP5)₂ only. Total RNA was extracted using the RNeasy kit (Quiagen) according to the manufacturer's protocol. mRNA was purified and RNAseq library was prepared using the TrueSeq RNA kit. Sequencing was performed on a Novaseq 6000 (Illumina). Reads were aligned to the human reference genome GRCh38.p10 using the STAR tool and transcripts quantified using the Kallisto program. Ensembl release 91 was used for the gene model definitions. For the heatmap and hierarchical clustering of significantly different genes the log 2fold changes in comparison to the mean of all samples was calculated and log 2fold changes >4 were set to 4. Gene set enrichment analysis was performed on Webgestalt.org (v2019)⁴¹. Input for GSEA were gene lists with the ensemble gene id and a ranking score (−log₁₀*p-value*sign (log₂ ratio)) as a metric for differential expression between two treatment groups. Differential expressed genes were compared to the gene ontology—biological process functional database and as a reference set the human genome—protein coding was used. For comparison of expression levels of selected genes the normalized gene count was used.

Data Analysis

Statistical analysis and graphs were prepared using GraphPad Prism (La Jolla, Calif.), unless otherwise stated. Data are expressed as mean+SD. Ordinary one-way ANOVA testing followed by post-hoc Dunnett's multiple comparison was used for comparison of treatment to control group. Image analysis was carried out using Matlab version R2018b and R2016b (MathWorks, Natick, Mass.) unless otherwise stated.

1. A compound comprising, particularly consisting of, two or more cyclohexanolpentakisester moieties described by a general formula (I) linked by a common central linker L 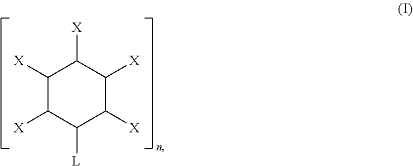
wherein each X is independently of any other X is selected from OPO₃²⁻, OPSO₂²⁻, OSO₃⁻ or CO₂⁻, L is a common central linker to which n individual moieties characterized by the formula in brackets are attached, and n is an integer from 2 to 10,
particularly wherein n is selected from 2, 3, 4, 6 and 8,
more particularly wherein n is selected from 2, 3 and 4,
wherein the common central linker L has a molecular weight <1000 g/mol, particularly <700 g/mol, more particularly <500 g/mol, or even <400 g/mol and wherein L comprises or essentially consists of a linear or branched poly(ethylene glycol) or polyglycerol,
and wherein optionally the linking moiety comprises a central core A selected from
an oxygen, nitrogen and/or fluorine substituted or unsubstituted C₁ to C₄ alkyl,
an oxygen, nitrogen and/or fluorine substituted or unsubstituted C₄ to C₇ cycloalkyl, or
an oxygen, nitrogen and fluorine substituted or unsubstituted five- or six-membered aryl.

2. The compound according to claim 1, wherein any one of the cyclohexanolpentakisester moieties described by a general formula (I) is independently selected from a moiety described by general formulae (Ta) or (Ib), 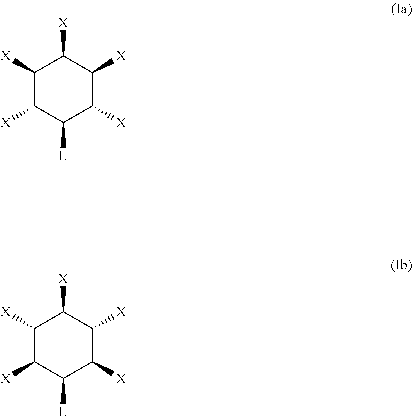
wherein L is a common central linker and X has the meaning defined in claim 1.

3. The compound according to claim 1, wherein the compound is characterized by a general formula (II), (IIi), (IIii), or (IIiii): 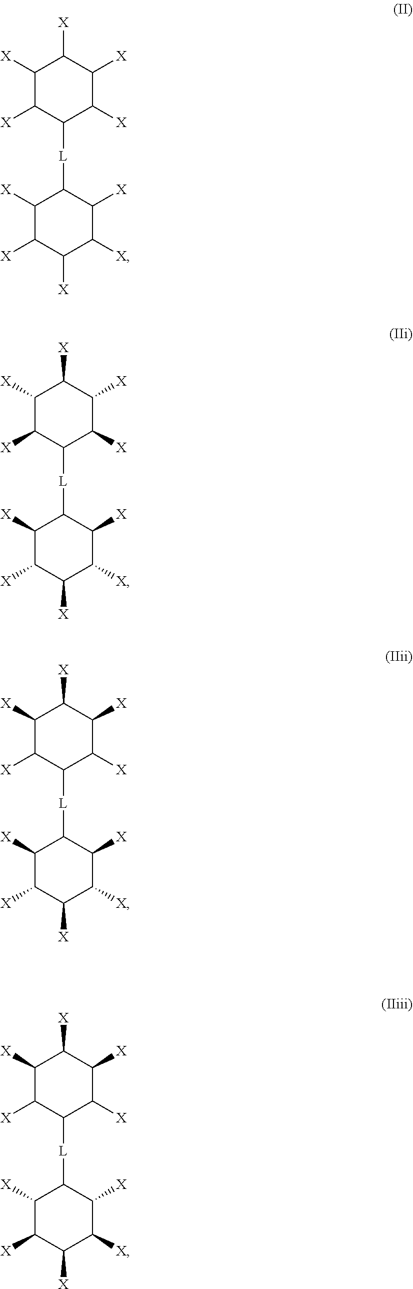
wherein each X is selected from OPO₃²⁻, OPSO₂²⁻, OSO₃⁻ or CO₂⁻ and wherein L is selected from —(O—CH₂—CH₂)ₘ—O— or —(O—CH₂—CH(OH)—CH₂)ₘ—O—, with m having a value between 1 and 12, particularly with m having a value between 2 and 8.

4. The compound according to claim 1, wherein the compound is characterized by formula (IIa), (IIb), (IIc), IId), (IIe), (IIf), (IIg), (IIh), (IIi), (IIk), (IIm), or (IIn) 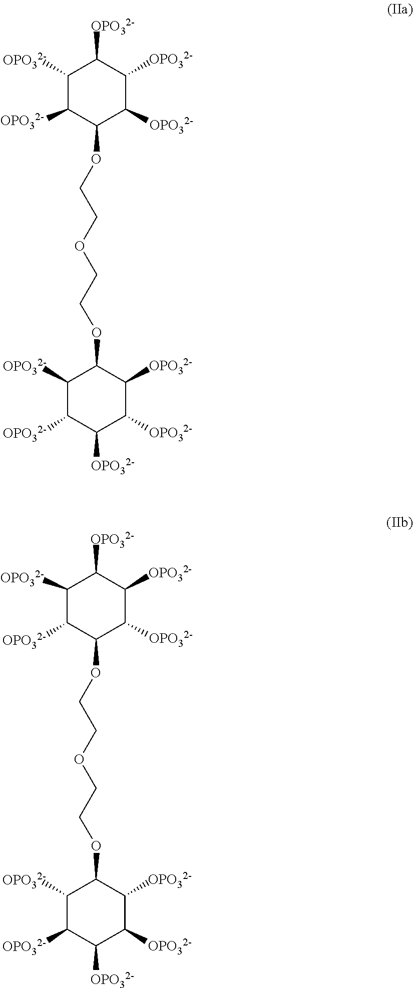 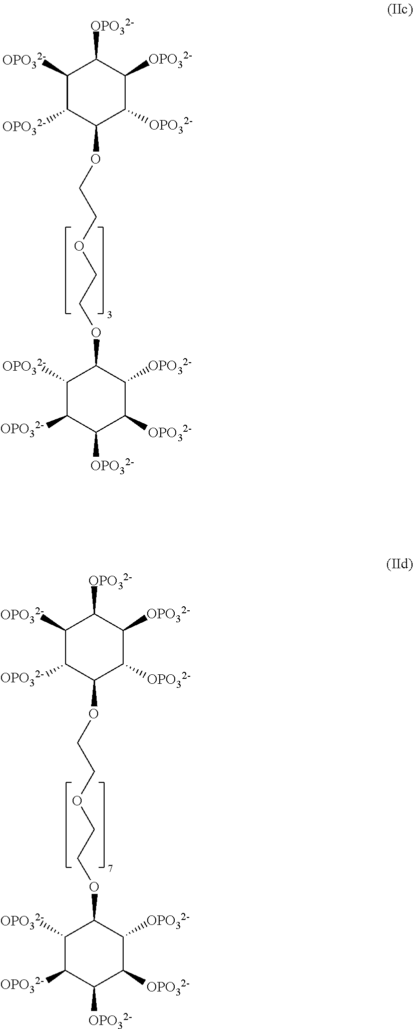 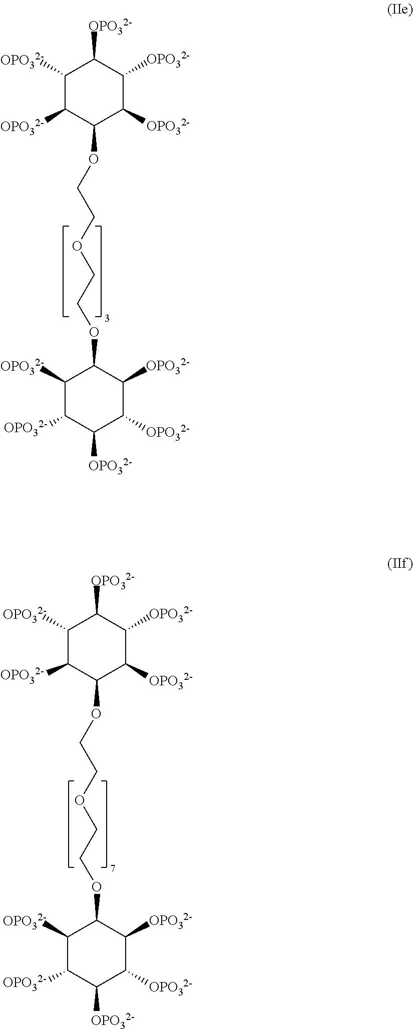 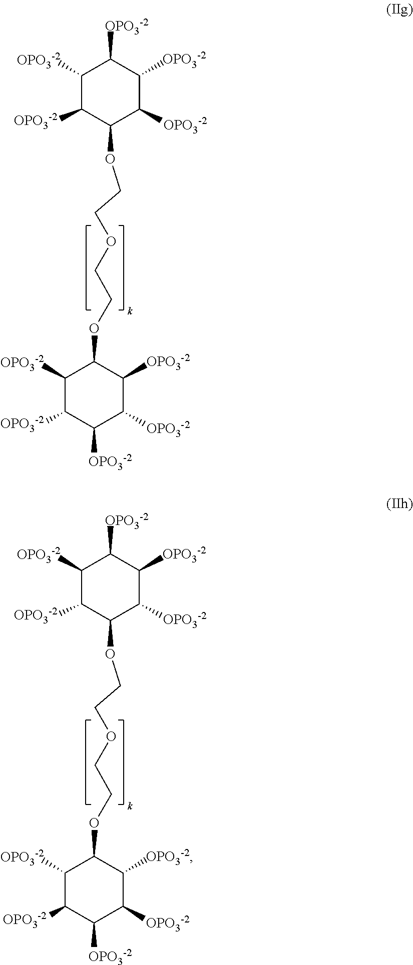 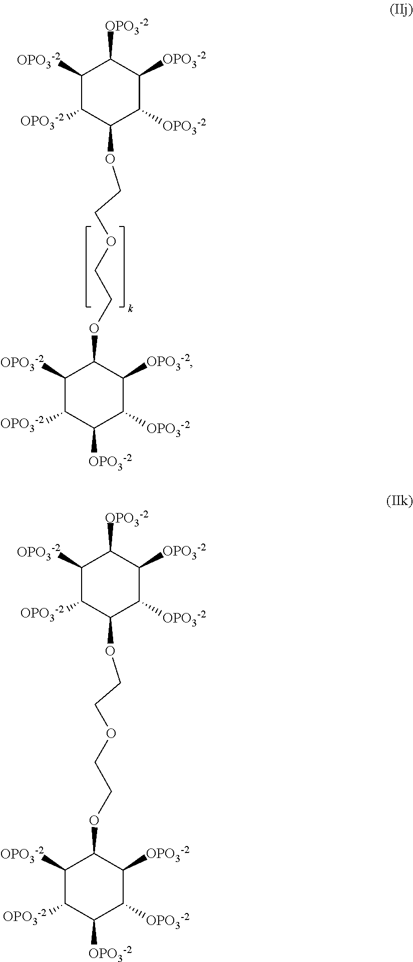 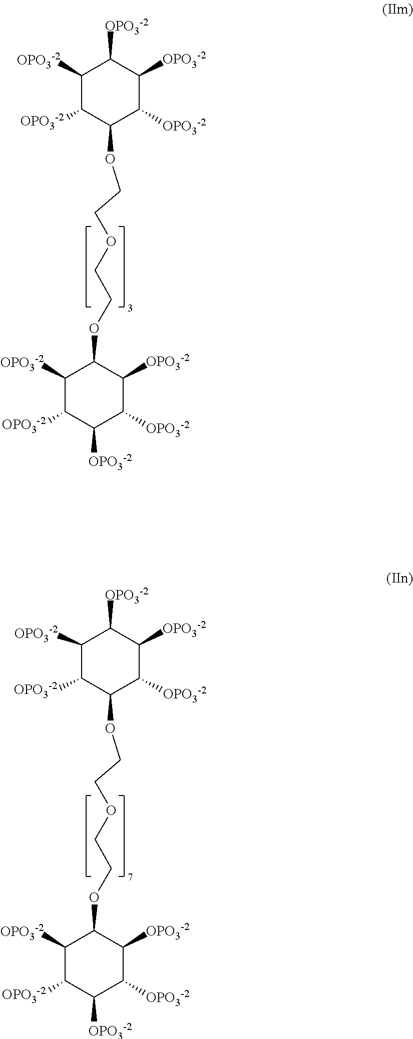
wherein k is an integer selected from 1, 2, 3, 4, 5, 6, 7, 8, 9, 10, 11 and 12.

5. The compound according to claim 1, wherein the compound is characterized by a general formula (III) 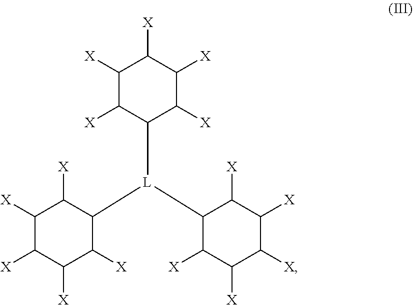
wherein each X has the meaning indicated above and
wherein the linking moiety L is 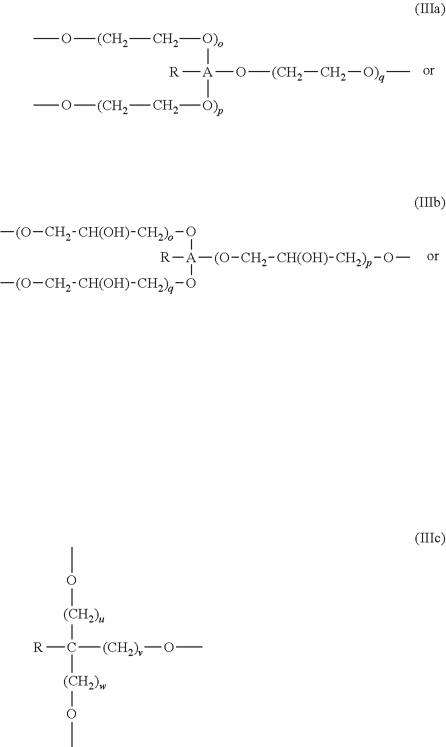
and wherein
o, p and q independently of each other have a value between 1 and 12 and the sum of o, p and q is ≤30,
particularly o, p and q independently of each other have a value between 2 and 4 and the sum of o, p and q is ≤12;
and
A is selected from a carbon atom (C), a hydroxy-, amino-, halogen- or carboxy-substituted or an unsubstituted C₄ to C₇ cycloalkyl moiety or a hydroxy-, amino-, halogen- or carboxy-substituted or an unsubstituted aryl and
R is selected from H and C₁-C₃ unsubstituted or N—, O and/or halogen substituted alkyl, particularly wherein R is H or unsubstituted alkyl or
u, v and w independently of each other have a value between 1 and 12, and the sum of u, v and w is ≤30, and R is selected from H and C₁-C₃ unsubstituted or N—, O and/or halogen substituted alkyl, particularly wherein R is H or unsubstituted alkyl,
particularly u, v and w independently of each other have a value between 2 and 4 and the sum u, v and w is ≤12.

6. The compound according to claim 5,
wherein the compound is characterized by formula (IIId) or (IIIe):

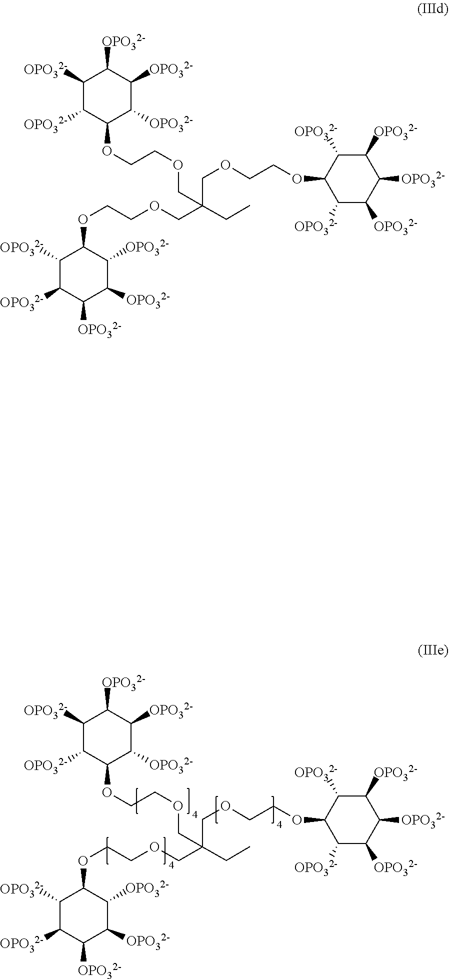

7. The compound according to claim 1, wherein the compound is characterized by a general formula (IV) 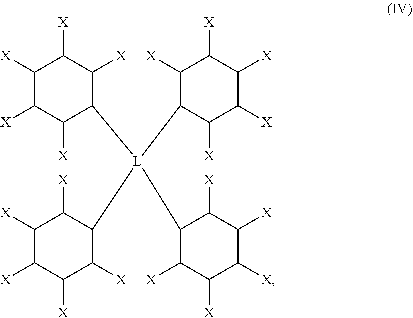
wherein each X has the meaning indicated above and
wherein the linking moiety L is 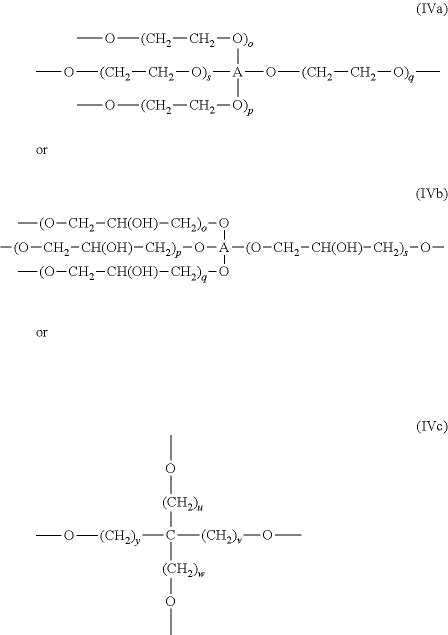
wherein
o, p, q and s independently of each other have a value between 1 and 12, and the sum of o, p, q and s is ≤40,
particularly wherein o, p, q and s independently of each other have a value between 2 and 4 and the sum of o, p, q and s is ≤12
and
A is a hydroxy-, amino-, halogen- or carboxy-substituted or an unsubstituted C₄ to C₇ cycloalkyl moiety or a hydroxy-, amino-, halogen- or carboxy-substituted or an unsubstituted aryl
or
u, v, w and y independently of each other have a value between 1 and 12 and the sum of u, v, w and y is ≤40,
particularly u, v, w and y independently of each other have a value between 2 and 4 and the sum of u, v, w and y is ≤12.

8. The compound according to claim 7,
wherein the compound is characterized by formula (IVd) or (IVe):

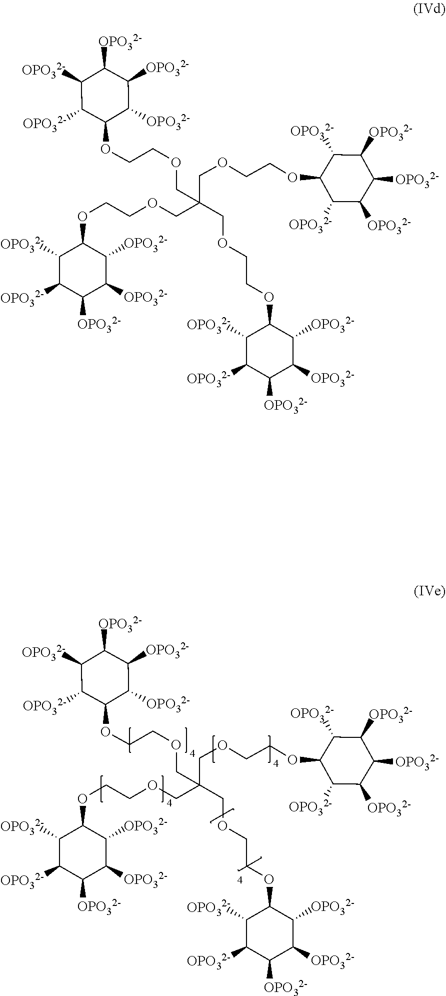

9. A compound according to claim 1 for use as a medicament.

10. A method for treatment of a condition related to pathological crystallization comprising administering to a subject in need thereof, the compound according to claim 1,
or a compound comprising, particularly consisting of, two or more cyclohexanolpentakisester moieties described by a general formula (I) linked by a common central linker L, 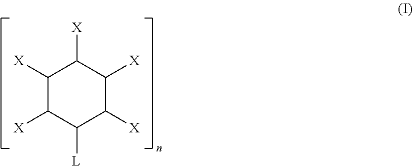
wherein X is selected from OPO₃²⁻, OPSO₂²⁻, or OSO₃⁻,
n is an integer from 2 to 10, particularly n is selected from 2, 3, 4, 6 and 8, particularly wherein n is selected from 2, 3 and 4,
wherein L is a linking moiety comprising or consisting of a linear or branched alkyl, optionally substituted by oxygen, nitrogen, (fluorine), particularly wherein the linking moiety has a molecular weight <1000 g/mol, particularly <700 g/mol, more particularly <500 g/mol, or even <400 g/mol.

11. The method according to claim 10, wherein said condition related to pathological crystallization is nephrocalcinosis or nephrolithiasis due to pathological calcium oxalate or phosphate crystallization in a patient, particularly wherein the condition is primary and secondary hyperoxaluria, Dent disease, Bartter's syndrome, distal renal tubule acidosis, neurogenic bladder, autosomal dominant polycystic kidney, calcium oxalate kidney stone formation, struvite stones and renal calcification.

12. The method according to claim 10, wherein the condition related to pathological crystallization is associated with formation of oxalate, phosphate and/or calcium precipitates.

13. The method c according to claim 10, wherein the compound is formulated for intravenous, intraperitoneal, intramuscular, intra-arterial, topical, intravesical or, particularly, subcutaneous administration.

14. A compound described by general formula Va, Vb, Vc, VIa, VIb or VIc, 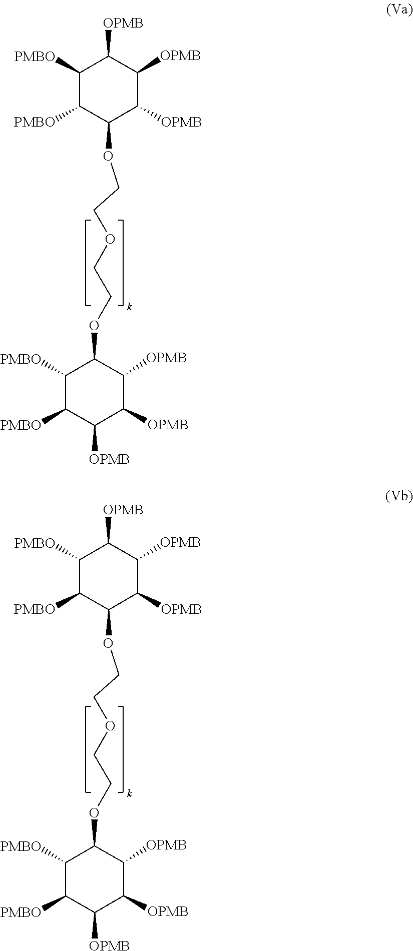 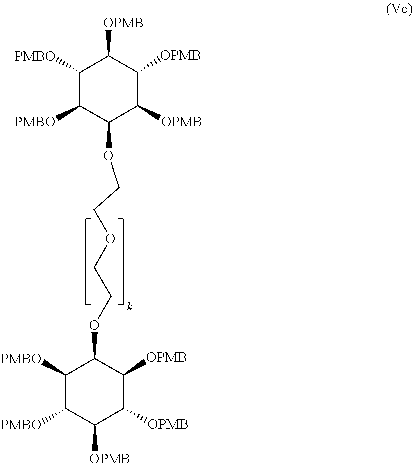 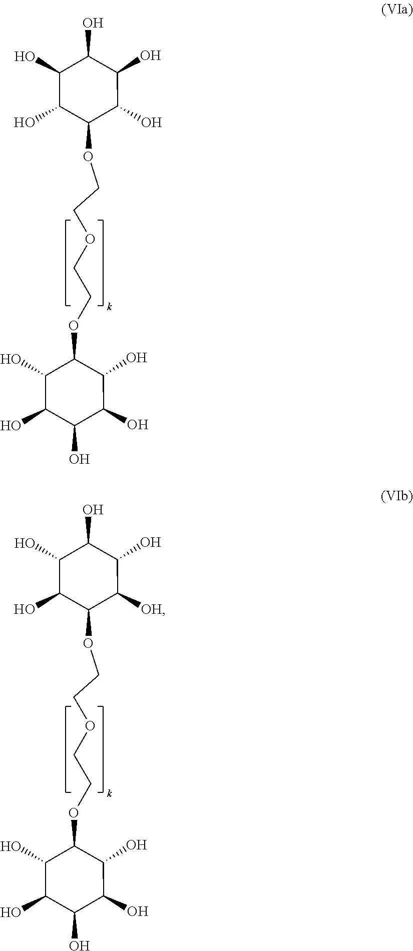 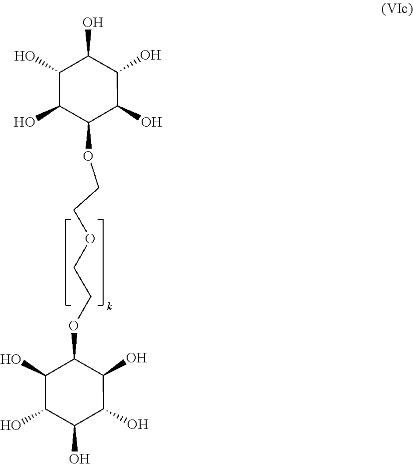
wherein OPMB signifies an O-(bis-paramethoxybenzyl)phosphate moiety and k is an integer selected from 1 to 12.

15. The compound according to claim 13, wherein k is selected from 2, 3, 4, 5, 6 or 7.
